SEQUENCE LISTING (26 sequences)

SEQ ID NO: 1 - DNA, 600 bp, Artificial Sequence
  Puromycin Resistance Gene
atgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 2 - protein, 199 aa, Artificial Sequence
  Puromycin Resistance Protein
MTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 3 - protein, 7 aa, Artificial Sequence
  Tobacco etch virus protease cleavage site
EXXYXQX

SEQ ID NO: 4 - protein, 7 aa, Artificial Sequence
  Tobacco etch virus protease cleavage site
ENLYFQG

SEQ ID NO: 5 - protein, 7 aa, Artificial Sequence
  Tobacco etch virus protease cleavage site
ENLYFQS

SEQ ID NO: 6 - protein, 19 aa, Artificial Sequence
  Exemplary self-cleaving polypeptide site comprising a 2A or 2A-like site.
LLNFDLLKLAGDVESNPGP

SEQ ID NO: 7 - protein, 19 aa, Artificial Sequence
  Exemplary self-cleaving polypeptide site comprising a 2A or 2A-like site.
TLNFDLLKLAGDVESNPGP

SEQ ID NO: 8 - protein, 14 aa, Artificial Sequence
  Exemplary self-cleaving polypeptide site comprising a 2A or 2A-like site.
LLKLAGDVESNPGP

SEQ ID NO: 9 - protein, 17 aa, Artificial Sequence
  Exemplary self-cleaving polypeptide site comprising a 2A or 2A-like site.
NFDLLKLAGDVESNPGP

SEQ ID NO: 10 - protein, 20 aa, Artificial Sequence
  Exemplary self-cleaving polypeptide site comprising a 2A or 2A-like site.
QLLNFDLLKLAGDVESNPGP

SEQ ID NO: 11 - protein, 24 aa, Artificial Sequence
  Exemplary self-cleaving polypeptide site comprising a 2A or 2A-like site.
APVKQTLNFDLLKLAGDVESNPGP

SEQ ID NO: 12 - protein, 40 aa, Artificial Sequence
  Exemplary self-cleaving polypeptide site comprising a 2A or 2A-like site.
VTELLYRMKRAETYCPRPLLAIHPTEARHKQKIVAPVKQT

SEQ ID NO: 13 - protein, 18 aa, Artificial Sequence
  Exemplary self-cleaving polypeptide site comprising a 2A or 2A-like site.
LNFDLLKLAGDVESNPGP

SEQ ID NO: 14 - protein, 40 aa, Artificial Sequence
  Exemplary self-cleaving polypeptide site comprising a 2A or 2A-like site.
LLAIHPTEARHKQKIVAPVKQTLNFDLLKLAGDVESNPGP

SEQ ID NO: 15 - protein, 33 aa, Artificial Sequence
  Exemplary self-cleaving polypeptide site comprising a 2A or 2A-like site.
EARHKQKIVAPVKQTLNFDLLKLAGDVESNPGP

SEQ ID NO: 16 - protein, 5 aa, Artificial Sequence
  Polypeptide linker sequence
DGGGS

SEQ ID NO: 17 - protein, 5 aa, Artificial Sequence
  Polypeptide linker sequence
TGEKP

SEQ ID NO: 18 - protein, 4 aa, Artificial Sequence
  Polypeptide linker sequence
GGRR

SEQ ID NO: 19 - protein, 5 aa, Artificial Sequence
  Polypeptide linker sequence
GGGGS

SEQ ID NO: 20 - protein, 14 aa, Artificial Sequence
  Polypeptide linker sequence
EGKSSGSGSESKVD

SEQ ID NO: 21 - protein, 18 aa, Artificial Sequence
  Polypeptide linker sequence
KESGSVSSEQLAQFRSLD

SEQ ID NO: 22 - protein, 8 aa, Artificial Sequence
  Polypeptide linker sequence
GGRRGGGS

SEQ ID NO: 23 - protein, 9 aa, Artificial Sequence
  Polypeptide linker sequence
LRQRDGERP

SEQ ID NO: 24 - protein, 12 aa, Artificial Sequence
  Polypeptide linker sequence
LRQKDGGGSERP

SEQ ID NO: 25 - protein, 16 aa, Artificial Sequence
  Polypeptide linker sequence
LRQKDGGGSGGGSERP

SEQ ID NO: 26 - DNA, 10 bp, Artificial Sequence
  Consensus Kozak sequence
gccrccatgg

(19) United States
(12) Patent Application Publication
(10) Pub. No.: US 2014/0199279 A1
(43) Pub. Date: Jul. 17, 2014

(54) METHODS FOR ENHANCING THE DELIVERY OF GENE-TRANSDUCED CELLS

(71) Applicants: Julian David Down, Cambridge, MA (US); Philippe Louis Leboulch, Paris (FR)

(72) Inventors: Julian David Down, Cambridge, MA (US); Philippe Louis Leboulch, Paris (FR)

(73) Assignee: Bluebird Bio, Inc., Cambridge, MA (US)

(21) Appl. No.: 13/978,338

(22) PCT Filed: Dec. 27, 2011
(86) PCT No.: PCT/US11/67347

§ 371 Date: Mar. 5, 2014

Related U.S. Application Data
(60) Provisional application No. 61/429,401, filed on Jan. 3, 2011.
(60) Provisional application No. 61/470,941, filed on Apr. 1, 2011.

(51) Int. Cl. C12N 15/86

(52) U.S. Cl. CPC C12N 15/86
USPC 424/93.21; 435/455

(57) ABSTRACT

The present invention provides novel methods for enhancing the delivery of transduced cells to a subject, which include both methods of selecting for transduced cells and methods of enhancing the reconstitution by transduced cells in a transplant recipient. The present invention further provides transfer vectors, including lentiviral vectors, useful in practicing the methods of the present invention. The methods and vectors of the present invention may be used in gene therapy of a variety of diseases and disorders, including but not limited to hematological diseases and disorders.

### CROSS REFERENCE TO RELATED APPLICATIONS

This application claims the benefit under 35 U.S.C. §119(e) of U.S. Provisional Application No. 61/429,401, filed Jan. 3, 2011, and U.S. Provisional Application No. 61/470,941, filed Apr. 1, 2011, each of which is incorporated by reference in its entirety.

### STATEMENT OF GOVERNMENT INTEREST

This invention was made with government support under Grant No. 1R43CA096457-1 awarded by the National Institutes of Health. The government has certain rights in this invention.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is BLBD_{—}001_{—}02_WO_ST25.txt. The text file is 10 KB, was created on Dec. 27, 2011, and is being submitted electronically via EFS-Web, concurrent with the filing of the specification.

### BACKGROUND

1. Technical Field

The present invention relates to methods for selecting gene-transduced multipotent cells, including stem cells, methods of enhancing the delivery of gene-transduced multipotent cells to transplant recipients, and methods for promoting the engraftment of gene-transduced multipotent cells in transplant recipients, as well as transfer vectors useful in practicing the methods of the present invention.

2. Description of the Related Art

Gene therapy via the ex vivo transduction of multipotent hematopoietic cells, including, e.g., hematopoietic stem cells (HSC), with a transfer vector that drives expression of a therapeutic polypeptide, followed by implantation of the resulting transduced cells into a transplant recipient, offers potential for the treatment of a variety of diseases and disorders, including genetic diseases of hematopoiesis and lymphopoiesis. However, the ability to achieve effective levels of therapeutic polypeptides can be limited by a number of factors, including the low frequency of the target multipotent cells, such as HSCs, within donor cell populations, the quiescent nature of the most primitive HSCs, unfavorable effects of in vitro cell culture on the engraftment potential of HSCs, and the presence of untransduced HSC in the transplanted cell population that compete with transduced HSC for engraftment and repopulation in the transplant recipient.

The ex vivo selection of cells that have been successfully modified genetically after exposure of cell populations to a given gene transfer vector remains an unmet goal of the field of gene therapy. Achieving this is essential in many instances to achieve potency and an appropriate risk/benefit ratio, when a given tissue must contains a large proportion of genetically modified cells, while the overall gene transfer efficiency is below the required threshold. Various ex vivo selection approaches that have been devised in the past have failed to show utility when primary cells, such as HSC, cannot withstand lengthy and/or traumatic physical manipulations. These include (i) fluorescence-activated cell sorting (FACS) or magnetic based approaches for the expression of a membrane marker co-expressed with the gene of interest and (ii) selection on the basis of co-expression of a dominant selectable marker that confers resistance to chemicals (e.g., G418, hygromycin). In particular, HSC are especially fragile in vitro and have resisted any attempt at ex vivo selection that would be practical for human clinical applications.

Examples of current methods for improving gene therapy via transplant of gene-engineered hematopoietic cells using a selective marker in retroviral vector includes the use of the O6-methylguanine-DNA-methyltransferase (MGMT) gene that confers resistance to agents with high guanine-O(6)alkylating potential, such as chloroethylnitrosoureas or temozolomide when delivered post-transplant in vivo (patent and refs.). Selective expansion of transduced hematopoietic stem cells has also been accomplished by incorporating the dihydrofolate reductase (DHFR) gene into the gene transfer vector selection for DHFR-expressing cells using post-transplant trimetrexate and nitrobenzylmercaptpurine riboside 5′ monophosphate (e.g., Zhang et al. 2005). These approaches, however, are limited by undesirable hematological toxicity from depletion of untransduced hematopoietic cells in vivo.

Another approach entails the pre-selection of cells ex vivo and prior to transplantation with consequent improvement of molecular chimerism in the recipient. This has been accomplished experimentally on the basis of expression of the green fluorescent protein using vectors that contain the green fluorescence protein (GFP) gene (e.g., Kalberer et al. 2000, Pawliuk et al., 1999) but is limited by the impracticality of isolating cells expressing fluorescent proteins for human use and the major loss of cells during the physical manipulation of the cells.

The selection of cells following retroviral gene transduction has also been performed experimentally on established cell lines using antibiotic resistance genes (e.g., against neomycin, hygromycin, puromycin) and adding the respective antibiotics. However, the selection by means of these antibiotics has been applied for many days in culture, usually 10 days at a minimum. Such lengthy culture of the cells in vitro is not compatible with sufficient maintenance of cell viability and state of differentiation of many primary cell types to be used in gene therapy protocols. Hence, hematopoietic stem cell populations submitted to this approach lose their engraftment potential in transplant recipients.

Another problem limiting the effectiveness of gene therapy using transduced HSCs is the occurrence of transient myelosuppression in transplant recipients who have received myeloablation prior to transplantation. These transplant recipients frequently suffer from myelosuppression due to the delay that exists following myeloablation and transplantation before the transduced HSCs sufficiently repopulate the transplant recipient's hematopoietic cell population.

Clearly, there is a need in the art for new methods of achieving high levels of transduced multipotent cells, including HSCs, as well as methods of inhibiting myelosuppression following transplantation of the transduced HSC. The present invention addresses this need by providing such methods, as well as transfer vectors useful in practicing these methods.

### BRIEF SUMMARY

The present invention includes novel methods of enhancing the reconstitution by transduced cells in a transplant recipient. In particular embodiments, these methods comprise puromycin-based selection of retrovirally/lentivirally transduced multipotent cells, which can effectively select fragile cells ex vivo with a sufficiently short length of exposure that results in both effectiveness and limited loss of multipotent cells. In addition, embodiments of the present invention are based on the development of a transplantation method that reduces or inhibits transient myelosuppression following myeloablation and subsequent transplantation. This method involves transplanting transduced multipotent cells capable of long-term repopulation, such as stem cells, in combination with cells capable of providing transient or short-term repopulation. In particular embodiments, the population of cells introduced to provide transient or short-term repopulation includes a higher percentage of cells having a reduced or negligible ability to achieve long-term repopulation as compared to the population of cells introduced to provide long-term repopulation, and may include progenitor cells and/or at least partially differentiated hematopoietic cells.

In one embodiment, the present invention provides a method of enhancing the reconstitution by transduced cells in a transplant recipient, which comprises selecting transduced cells prior to transplantation into said transplant recipient, wherein said transduced cells are selected by a method comprising: (i) contacting in vitro a first population of cells comprising multipotent cells, including stem cells, with a transfer vector comprising a polynucleotide sequence encoding a puromycin resistance polypeptide operably linked to a promoter sequence, thereby generating a second population of cells comprising transduced multipotent cells, including stem cells; and (ii) contacting in vitro said second population of cells with puromycin at a concentration of 1-25 μg/ml for 4 days or less, thereby generating a third population of cells comprising transduced multipotent cells, including stem cells, wherein said third population of cells comprises a higher percentage of transduced multipotent cells than said second population of cells, and wherein said third population of cells is capable of sustaining the production of at least two distinct cell lineages containing said transfer vector for a duration of at least four months in vivo after transplantation of said third population of cells into a transplant recipient. In certain embodiments, the third population of cells includes at least 50%, at least 60%, at least 70%, at least 80%, or at least 90% transduced cells. In particular embodiments, the method further comprises transplanting a plurality of said third population of cells into said transplant recipient. In certain embodiments, the first population of cells was obtained from said transplant recipient. In certain embodiments, the first population of cells was obtained from bone marrow, peripheral mobilized blood, cord blood and/or embryonic stem cells. In particular embodiments, the at least four months may occur at any time beginning within two years of said transplantation. Accordingly, there may be a lag period between transplantation and when the implanted, transduced cells begin sustained production of the at least two distinct cell lineages. In one embodiment, said second population of cells is contacted with about 5 μg/ml puromycin for about 24 hours. In certain embodiments, said first population of cells comprises hematopoietic stem cells. In particular embodiments, said transfer vector further comprises a polynucleotide sequence encoding a therapeutic polypeptide operably linked to a promoter sequence. In particular embodiments, said transfer vector is a retroviral vector. In various embodiments, said transfer vector is a lentiviral vector. In various embodiments, said lentiviral vector is a human immunodeficiency virus (HIV) vector, a simian immunodeficiency virus (SIV) vector, or an equine infectious anaemia virus (EIAV) vector. In particular embodiments, said transfer vector is a transposon.

In particular embodiments of methods of the present invention, the polynucleotide encoding the puromycin resistance polypeptide and the polynucleotide encoding the therapeutic polypeptide are operably linked to the same promoter sequence. In certain embodiments, the polynucleotide encoding the puromycin resistance polypeptide and the polynucleotide encoding the therapeutic polypeptide are operably linked to different promoter sequences. In certain embodiments, the promoter or promoters are constitutive promoters. In related embodiments, the promoter sequence operably linked to the polynucleotide encoding the puromycin resistance polypeptide is selected from the group consisting of: a constitutive promoter, an inducible promoter, and a tissue specific promoter. In certain embodiments, said promoter is a tissue specific promoter that has greater activity in stem cells as compared to its activity in cells differentiated from said stem cells. In particular embodiments, said stem cells are hematopoietic stem cells. In certain embodiments, the promoter sequence operably linked to the polynucleotide encoding the therapeutic polypeptide is selected from the group consisting of: a constitutive promoter, an inducible promoter, and a tissue specific promoter. In certain embodiments, the promoter is a tissue specific promoter that has reduced activity in multipotent cells as compared to its activity in cells differentiated from said multipotent cells. In particular embodiments, said tissue specific promoter is active in red blood cells.

In certain embodiments of methods of the present invention, said transfer vector further comprises a polynucleotide comprising a suicide gene or cDNA operably linked to a promoter sequence, wherein said suicide gene or cDNA encodes a suicide polypeptide. In certain embodiments, said suicide gene or cDNA encodes a thymidine kinase derivative. In particular embodiments, said suicide gene or cDNA encodes a thymidylate kinase (TmpK) or derivative thereof. In certain embodiments, said suicide gene or cDNA encodes a caspase or derivative thereof. In particular embodiments, the polynucleotide sequence comprising the suicide gene or cDNA is not operatively linked to a promoter sequence present in the transfer vector. In other embodiments, the polynucleotide sequence comprising the suicide gene or cDNA is operatively linked to a promoter sequence present in the transfer vector. In particular embodiments, the promoter sequence present in the transfer vector and operatively linked to the polynucleotide sequence comprising the suicide gene or cDNA is an inducible promoter. In certain embodiments, the polynucleotide sequence comprising the suicide gene or cDNA and the polynucleotide sequence encoding the therapeutic polypeptide are present in the transfer vector in opposite orientations. In particular embodiments, said transfer vector comprises a splice acceptor sequence upstream of the suicide gene or cDNA. In particular embodiments, the polynucleotide sequence comprising the suicide gene or cDNA comprises a Kozak consensus sequence at the 5′ end of the suicide gene or cDNA and a transcription terminator sequence 3′ of the suicide gene or cDNA. In particular embodiments, the transfer vector expresses said puromycin resistance polypeptide and said suicide polypeptide as an in-frame fusion polypeptide. In particular embodiments, the fusion polypeptide is a direct fusion of the puromycin resistance polypeptide and the suicide polypeptide. In certain embodiments, said puromycin resistance polypeptide and said suicide polypeptide are expressed by use of an internal ribosome entry site (IRES) present in said transfer vector, wherein the IRES may be located between the polynucleotide sequence encoding the puromycin resistance polypeptide and the polynucleotide sequence comprising the suicide gene or cDNA. In certain embodiments, said puromycin resistance polypeptide and said suicide polypeptide are expressed by use of a translational 2A signal sequence present in said transfer vector. In certain embodiments, the fusion polypeptide comprises a linker sequence between the puromycin resistance polypeptide and the suicide polypeptide. In particular embodiments, the linker sequence comprises a Gly3 linker sequence. In particular embodiments, the linker sequence comprises an autocatalytic peptide cleavage site. In particular embodiments, the autocatalytic peptide cleavage site comprises a translational 2A signal sequence. In some embodiments, the transfer vector comprises a polynucleotide sequence encoding junk sequence between the polynucleotide sequence encoding the puromycin resistance polypeptide and the polynucleotide sequence comprising the suicide gene or cDNA. In particular embodiments, the polynucleotide sequence encoding junk sequence is flanked by a stop codon at its 5′ end and a start codon at its 3′ end.

In particular embodiments of methods of the present invention, said methods further comprise providing said third population of cells to a subject in combination with a fourth population of cells, said fourth population of cells comprising progenitor cells, wherein said fourth population of cells is capable of providing transient or short term hematopoietic support after transplantation of said fourth population of cells into a transplant recipient. In certain embodiments, said fourth population of cells was previously exposed to conditions that induce expansion and/or at least partial differentiation of multipotent cells. In particular embodiments, said fourth population of cells is not transduced. In other embodiments, said fourth population of cells comprises cells transduced and selected by a method comprising: (i) contacting the fourth population of cells with a transfer vector comprising a polynucleotide sequence encoding a puromycin resistance polypeptide operably linked to a promoter sequence; and (ii) contacting the fourth population of cells with puromycin at a concentration of 1-25 μg/ml for 4 days or less, thereby selecting for transduced cells comprising the puromycin resistance polypeptide. In certain embodiments, said fourth population of cells comprises hematopoietic cells. In certain embodiments, said first and fourth population of cells were obtained from the same subject. In related embodiments, said first and fourth population of cells were obtained from bone marrow, peripheral mobilized blood, cord blood, and/or embryonic stem cells.

In particular embodiments of methods of the present invention, said methods further comprise contacting at least one of said first, second or third population of cells with one or more agents capable of increasing the number of stem cells present in the contacted cell population. In particular embodiments, said one or more agents comprise an aryl hydrogen receptor antagonist. In one embodiment, said aryl hydrogen receptor antagonist comprises SR1. In certain embodiments, said one or more agents comprise a combination of growth factors. In particular embodiments, said multipotent cells are increased in number following a culture period of between 4 and 21 days. In certain embodiments, at least 75% of said third population of cells are transduced.

### BRIEF DESCRIPTION OF THE SEVERAL VIEWS OF THE DRAWINGS

FIG. 1 provides a schematic diagram of a representative HIV transfer vector (HPV654) of the invention, which includes nucleic acid sequences encoding the puromycin resistance gene (PURO) operably linked to the constitutive pgk promoter (PGK), and a therapeutic human β-globin polypeptide (human β-globingene) operably linked to β-globin locus control region sequences (β-LCR).

FIG. 2 provides diagrams of puromycin selection of transduced cells. FIG. 2A provides a schematic diagram depicting puromycin selection of bone marrow or G-CSF mobilized peripheral blood CD34⁺ cells transduced using a transfer vector that confers puromycin resistance (HPV654) at either 10% (left diagram) or 50% (right diagram) supernatants. As shown, treatment of the bone marrow CD34⁺ cells transduced with HPV654 (10%) with 5 μg/ml of puromycin for 24 hours resulted in the selection of 100% transduced cells after 14 days growth, as compared to only 23% transduced cells in the absence of puromycin selection. Treatment of the cells transduced with HPV654 (50%) with 5 μg/ml of puromycin for 24 hours resulted in the selection of 79% transduced cells after 14 days growth, as compared to only 16% transduced cells in the absence of puromycin selection. Similarly, G-CSF mobilized peripheral blood CD34⁺ cells transduced with HPV654 (10%) with 5 μg/ml of puromycin for 24 hours resulted in the selection of 88% transduced cells, as compared to 55% transduced cells in the absence of puromycin selection (FIG. 2B). Treatment of the cells transduced with HPV654 (50%) with 5 μg/ml of puromycin for 24 hours resulted in the selection of 100% transduced cells, as compared to only 37% transduced cells in the absence of puromycin selection.

FIG. 3 provides a schematic diagram depicting a process of the present invention for selecting transduced cell prior to transplantation into a recipient. Untransduced cells obtained from a donor (first population), which include hematopoietic multipotent stem cells, are transduced using a transfer vector that confers puromycin resistance and encodes a therapeutic polypeptide, resulting in a fraction of the multipotent stem cells being transduced (second population). Following transduction, the cells are contacted with puromycin, which removes untransduced cells, leaving transduced cells that include transduced multipotent stem cells (third population). These transduced cells are transplanted into a recipient, where the transduced multipotent cells grow without competition from untransduced cells and eventually reconstitute a cell population within the recipient.

FIG. 4 provides schematic diagrams showing embodiments of methods of the present invention for improving hematopoietic reconstitution that include: (A) the addition of expanded progenitors capable of only transient repopulation (fourth population) to puromycin-selected transduced cells, transduced with a transfer vector that confers puromycin resistance, and capable of long-term repopulation in the transplanted host (third population); and (B) the expansion of the selected transduced cells by culturing them in the presence of an agent that promotes the expansion of HSCs. These expanded and transduced cells are transplanted into a recipient, where the transduced progenitor and stem cells grow without competition from untransduced cells and eventually provide improved reconstitution within the recipient. The graphs at the bottom of FIGS. 4A and 4B show the level of myelosuppression over time following transplant into a recipient after myeloablation (left graph), and the repopulation from transplanted cells over time following transplant into the recipient after myeloablation (right graph).

### DETAILED DESCRIPTION

The present invention is based, in part, on the unexpected discovery that puromycin-based selection of retrovirally/lentivirally transduced hematopoietic cells can effectively select fragile cells, such as stem cells, ex vivo with a sufficiently short length of exposure that results in both effectiveness and limited loss of cells. This is exemplified herein by gene transfer to hematopoietic stem cells. However, this approach is applicable to other cell types for which the ex vivo selection of fragile cells is desirable to achieve increased therapeutic potency. In addition, aspects of the present invention are based on the development of a transplantation method that reduces or inhibits transient myelosuppression following myeloablation and subsequent transplantation. This method involves transplanting transduced multipotent cells capable of repopulation, such as stem cells, in combination with untransduced cells having a comparatively reduced or negligible ability to achieve repopulation, such as progenitor cells or at least partially differentiated hematopoietic cells. The progenitor cells or at least partially differentiated hematopoietic cells transiently repopulate the transplant recipient, thus inhibiting myelosuppression, while the transduced stem cells undergo the longer process of long-term repopulation. This method is particularly effective when transduced cells have undergone selection, since there will typically be a reduced number of cells being transplanted as compared to when transduced cells are not selected, so the transplant recipient is at increased risk of myelosuppression.

Accordingly, the present invention addresses an unmet clinical need for improving the efficacy of gene therapy in the treatment of genetic diseases, whereby only a portion of cells have been effectively targeted by a transfer vector and at levels that are insufficient for conferring a therapeutic effect. The invention specifically relates to the enrichment and selection of genetically engineered cells from a mixed population of cells, where removal of untransduced (e.g., uncorrected) cells is the desired outcome.

### DEFINITIONS

As used herein, the following terms and phrases used to describe the invention shall have the meanings provided below.

The term “retrovirus” refers to any known retrovirus (e.g., type c retroviruses, such as Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend, Murine Stem Cell Virus (MSCV) and Rous Sarcoma Virus (RSV)). “Retroviruses” of the invention also include human T cell leukemia viruses, HTLV-1 and HTLV-2, and the lentiviral family of retroviruses, such as Human Immunodeficiency Viruses, HIV-1, HIV-2, simian immunodeficiency virus (SIV), feline immunodeficiency virus (FIV), equine immunodeficiency virus (EIV), and other classes of retroviruses.

Retroviruses are RNA viruses that utilize reverse transcriptase during their replication cycle. The retroviral genomic RNA is converted into double-stranded DNA by reverse transcriptase. This double-stranded DNA form of the virus is capable of being integrated into the chromosome of the infected cell; once integrated, it is referred to as a “provirus.” The provirus serves as a template for RNA polymerase II and directs the expression of RNA molecules which encode the structural proteins and enzymes needed to produce new viral particles.

At each end of the provirus are structures called “long terminal repeats” or “LTRs.” The term “long terminal repeat (LTR)” refers to domains of base pairs located at the ends of retroviral DNAs which, in their natural sequence context, are direct repeats and contain U3, R and U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals and sequences needed for replication and integration of the viral genome. The viral LTR is divided into three regions called U3, R and U5. The U3 region contains the enhancer and promoter elements. The U5 region is the sequence between the primer binding site and the R region and contains the polyadenylation sequence. The R (repeat) region is flanked by the U3 and U5 regions. The LTR composed of U3, R and U5 regions, appears at both the both the 5′ and 3′ ends of the viral genome. In one embodiment of the invention, the promoter within the LTR, including the 5′ LTR, is replaced with a heterologous promoter. Examples of heterologous promoters which can be used include, for example, the cytomegalovirus (CMV) promoter.

The term “lentivirus” refers to a group (or genus) of retroviruses that give rise to slowly developing disease. Viruses included within this group include HIV (human immunodeficiency virus; including HIV type 1, and HIV type 2), the etiologic agent of the human acquired immunodeficiency syndrome (AIDS); visna-maedi, which causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which cause immune deficiency and encephalopathy in sub-human primates. Diseases caused by these viruses are characterized by a long incubation period and protracted course. Usually, the viruses latently infect monocytes and macrophages, from which they spread to other cells. HIV, FIV, and SIV also readily infect T lymphocytes (i.e., T-cells).

The term “hybrid” refers to a vector, LTR or other nucleic acid containing both lentiviral sequences and non-lentiviral retroviral sequences.

The term “vector” or “transfer vector” refers to a nucleic acid molecule capable of transporting another nucleic acid to which it has been linked. The term “expression vector” includes any vector, (e.g., a plasmid, cosmid or phage chromosome) containing a gene construct in a form suitable for expression by a cell (e.g., linked to a promoter). In the present specification, “plasmid” and “vector” are used interchangeably, as a plasmid is a commonly used form of vector. Moreover, the invention is intended to include other vectors which serve equivalent functions.

The term “viral vector” refers to a vector containing structural and functional genetic elements that are primarily derived from a virus.

The term “retroviral vector” refers to a vector containing structural and functional genetic elements that are primarily derived from a retrovirus.

The term “lentiviral vector” refers to a vector containing structural and functional genetic elements outside the LTRs that are primarily derived from a lentivirus.

The term “self-inactivating vector” (SIN vector) refers to vectors, e.g., retroviral or lentiviral vectors, in which the right (3′) LTR enhancer-promoter region, known as the U3 region, has been modified (e.g., by deletion or substitution) to prevent viral transcription beyond the first round of viral replication. Consequently, the vectors are capable of infecting and then integrating into the host genome only once, and cannot be passed further. This is because the right (3′) LTR U3 region is used as a template for the left (5′) LTR U3 region during viral replication and, thus, the viral transcript cannot be made without the U3 enhancer-promoter. If the viral transcript is not made, it cannot be processed or packaged into virions, hence the life cycle of the virus ends. Accordingly, SIN vectors greatly reduce risk of creating unwanted replication-competent virus since the right (3′) LTR U3 region has been modified to prevent viral transcription beyond the first round of replication, hence eliminating the ability of the virus to be passed.

The term “TAR” refers to the “trans-activation response” genetic element located in the R region of lentiviral (e.g., HIV) LTRs. This element interacts with the lentiviral trans-activator (tat) genetic element to enhance viral replication.

The term “R region” refers to the region within retroviral LTRs beginning at the start of the capping group (i.e., the start of transcription) and ending immediately prior to the start of the poly A tract. The R region is also defined as being flanked by the U3 and U5 regions. The R region plays an important role during reverse transcription in permitting the transfer of nascent DNA from one end of the genome to the other.

The term “transfection” refers to the introduction of foreign DNA into eukaryotic cells. Transfection may be accomplished by a variety of means known in the art including but not limited to calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “transduction” refers to the delivery of a gene(s) or other polynucleotide sequence using a viral or retroviral vector by means of viral infection rather than by transfection. In preferred embodiments, retroviral vectors are transduced by packaging the vectors into virions prior to contact with a cell. For example, an anti-HIV gene carried by a retroviral vector can be transduced into a cell through infection and provirus integration. In certain embodiments, a cell is “transduced” if it comprises a gene or other polynucleotide sequence delivered to the cell by infection using a viral or retroviral vector. In particular embodiments, a transduced cell comprises the gene or other polynucleotide sequence delivered to by a viral or retroviral vector in its cellular genome.

The term “promoter/enhancer” refers to a segment of DNA which contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

Efficient expression of recombinant DNA sequences in eukaryotic cells requires expression of signals directing the efficient termination and polyadenylation of the resulting transcript. Transcription termination signals are generally found downstream of the polyadenylation signal. The term “poly A site” or “poly A sequence” as used herein denotes a DNA sequence which directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be “heterologous” or “endogenous.” An endogenous poly A signal is one that is found naturally at the 3′ end of the coding region of a given gene in the genome. A heterologous poly A signal is one which is one which is isolated from one gene and placed 3′ of another gene.

The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al. (1991) J. Virol. 65: 1053; and Cullen et al. (1991) Cell 58: 423), and the hepatitis B virus post-transcriptional regulatory element (PRE) (see, e.g., Huang et al. (1995) Molec. and Cell. Biol. 15(7): 3864; Huang et al. (1994) J. Virol. 68(5): 3193; Huang et al. (1993) Molec. and Cell. Biol. 3(12): 7476), and U.S. Pat. No. 5,744,326). Generally, the RNA export element is placed within the 3′ UTR of a gene, and can be inserted as one or multiple copies. RNA export elements can be inserted into any or all of the separate vectors generating the packaging cell lines of the present invention.

As used herein, the term “packaging cell lines” is used in reference to cell lines that do not contain a packaging signal, but do stably or transiently express viral structural proteins and replication enzymes (e.g., gag, pol and env) which are necessary for the correct packaging of viral particles.

The phrase “retroviral packaging cell line” refers to a cell line (typically a mammalian cell line) which contains the necessary coding sequences to produce viral particles which lack the ability to package RNA and produce replication-competent helper-virus. When the packaging function is provided within the cell line (e.g., in trans by way of a plasmid vector), the packaging cell line produces recombinant retrovirus, thereby becoming a “retroviral producer cell line.”

The term “nucleic acid cassette” as used herein refers to genetic sequences within the vector which can express a RNA, and subsequently a protein. The nucleic acid cassette contains the gene of interest. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. In a preferred embodiment of the invention, the nucleic acid cassette contains the sequence of a therapeutic gene used to treat a hemoglobinopathic condition. The cassette can be removed and inserted into a vector or plasmid as a single unit.

As used herein, the term “gene of interest” refers to the gene inserted into the polylinker of an expression vector. In certain embodiments, the gene of interest encodes a polypeptide that provides a therapeutic effect in the treatment or prevention of a disease or disorder, which may be referred to as a “therapeutic polypeptide.” In one embodiment, the gene of interest encodes a gene which provides a therapeutic function for the treatment of a hemoglobinopathy. Genes of interest, and polypeptides encoded therefrom, include both wild-type genes and polypeptides, as well as functional variants and fragments thereof. In particular embodiments, a functional variant has at least 80%, at least 90%, at least 95%, or at least 99% identity to a corresponding wild-type reference polynucleotide or polypeptide sequence. In certain embodiments, a functional variant or fragment has at least 50%, at least 60%, at least 70%, at least 80%, or at least 90% of a biological activity of a corresponding wild-type polypeptide.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 97%, 98%, 99% or 100% sequence identity to any of the reference sequences described herein (see, e.g., Sequence Listing), typically where the polypeptide variant maintains at least one biological activity of the reference polypeptide.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

The term “promoter” as used herein refers to a recognition site of a DNA strand to which the RNA polymerase binds. The promoter forms an initiation complex with RNA polymerase to initiate and drive transcriptional activity. The complex can be modified by activating sequences termed “enhancers” or inhibitory sequences termed “silencers”.

As used herein, the term “cis” is used in reference to the presence of genes on the same chromosome. The term “cis-acting” is used in reference to the controlling effect of a regulatory gene on a gene present on the same chromosome. For example, promoters, which affect the synthesis of downstream mRNA are cis-acting control elements.

The term “suicide gene” is used herein to define any gene that expresses a product that is fatal to the cell expressing the suicide gene. In one embodiment, the suicide gene is cis-acting in relation to the gene of interest on the vector of the invention, Examples of suicide genes are known in the art, including HSV thymidine kinase (HSV-Tk).

The term “operably linked”, refers to a juxtaposition wherein the components described are in a relationship permitting them to function in their intended manner. In one embodiment, the term refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The terms “pseudotype” or “pseudotyping” as used herein, refer to a virus whose viral envelope proteins have been substituted with those of another virus possessing preferable characteristics. For example, HIV can be pseudotyped with vesicular stomatitis virus G-protein (VSV-G) envelope proteins, which allows HIV to infect a wider range of cells because HIV envelope proteins (encoded by the env gene) normally target the virus to CD4+ presenting cells. In a preferred embodiment of the invention, lentiviral envelope proteins are pseudotyped with VSV-G.

As used herein, the term “packaging” refers to the process of sequestering (or packaging) a viral genome inside a protein capsid, whereby a virion particle is formed. This process is also known as encapsidation. As used herein, the term “packaging signal” or “packaging sequence” refers to sequences located within the retroviral genome which are required for insertion of the viral RNA into the viral capsid or particle. Several retroviral vectors use the minimal packaging signal (also referred to as the psi [ψ] sequence) needed for encapsidation of the viral genome. Thus, as used herein, the terms “packaging sequence,” “packaging signal,” “psi” and the symbol “ψ” are used in reference to the non-coding sequence required for encapsidation of retroviral RNA strands during viral particle formation.

As used herein, the term “replication-defective” refers to virus that is not capable of complete, effective replication such that infective virions are not produced (e.g., replication-defective lentiviral progeny). The term “replication-competent” refers to wild-type virus or mutant virus that is capable of replication, such that viral replication of the virus is capable of producing infective virions (e.g., replication-competent lentiviral progeny).

As used herein, the term “incorporate” refers to uptake or transfer of a vector (e.g., DNA or RNA) into a cell such that the vector can express a therapeutic gene product within the cell. Incorporation may involve, but does not require, integration of the DNA expression vector or episomal replication of the DNA expression vector.

As used herein, the term “erythroid-specific expression” or “red blood cell-specific expression” refers to gene expression which only occurs in erythrocytes or red blood cells (RBCs), used interchangeably herein.

The term “gene delivery” or “gene transfer” refers to methods or systems for reliably inserting foreign DNA into target cells, such as into muscle cells. Such methods can result in transient or long term expression of genes. Gene transfer provides a unique approach for the treatment of acquired and inherited diseases. A number of systems have been developed for gene transfer into mammalian cells. See, e.g., U.S. Pat. No. 5,399,346. The lentiviral vector of the invention is optimized to express antisickling proteins at therapeutic levels in virtually all circulating RBCs.

The term “stem cell” refers to a multipotent cell from which a progenitor cell is derived. Stem cells are defined by their ability to self-renew. Stem cells include, for example, embryonic stem cells and somatic stem cells. Hematopoietic stem cells can generate daughter cells of any of the hematopoietic lineages. Stem cells with long term hematopoietic reconstituting ability can be distinguished by a number of physical and biological properties from differentiated cells and progenitor cells (see, e.g., Hodgson, G. S. & Bradley, T. R., Nature, Vol. 281, pp 381-382; Visser et al., J. Exp. Med., Vol. 59, pp. 1576-1590, 1984; Spangrude et al., Science, Vol. 241, pp. 58-62, 1988; Szilvassy et al., Blood, Vol. 74, pp. 930-939, 1989; Ploemacher, R. E. & Brons, R. H. C., Exp. Hematol., Vol. 17, pp. 263-266, 1989). Certain hematopoietic stem cells have the capacity to provide long-term reconstitution of a hematopoietic cell population in a transplant recipient. Multipotent cells have the capacity to differentiate into two or more different cells.

As used herein, the term “progenitor” or “progenitor cells” refers to cells which are the precursors of differentiated cells. Many progenitor cells differentiate along a single lineage, but may have quite extensive proliferative capacity. Examples of progenitor cells include, but are not limited to, hematopoietic progenitor cells, myeloid progenitor cells, and lymphoid progenitor cells. Hematopoietic progenitor cells are not self-renewing but have the capacity to provide transient or short-term reconstitution of a hematopoietic cell population in a transplant recipient.

The term “globin” is used here to mean all proteins or protein subunits that are capable of covalently or noncovalently binding a heme moiety, and can therefore transport or store oxygen. Subunits of vertebrate and invertebrate hemoglobins, vertebrate and invertebrate myoglobins or mutants thereof are included by the term globin. Examples of globins include β-globin or variant thereof, β-globin or variant thereof, a β-globin or a variant thereof, and β-globin.

As used herein, “hematopoiesis,” refers to the formation and development of blood cells from progenitor cells as well as formation of progenitor cells from stem cells. Blood cells include but are not limited to erythrocytes or red blood cells (RBCs), reticulocytes, monocytes, neutrophils, megakaryotes, eosinophils, basophils, B-cells, macrophages, granulocytes, mast cells, thrombocytes, and leukocytes.

As used herein, the term “hemoglobinopathy” or “hemoglobinopathic condition” includes any disorder involving the presence of an abnormal hemoglobin molecule in the blood. Examples of hemoglobinopathies included, but are not limited to, hemoglobin C disease, hemoglobin sickle cell disease (SCD), sickle cell anemia, and thalassemias. Also included are hemoglobinopathies in which a combination of abnormal hemoglobins are present in the blood (e.g., sickle cell/Hb-C disease).

The term “sickle cell anemia” or “sickle cell disease” is defined herein to include any symptomatic anemic condition which results from sickling of red blood cells. Manifestations of sickle cell disease include: anemia; pain; and/or organ dysfunction, such as renal failure, retinopathy, acute-chest syndrome, ischemia, priapism and stroke. As used herein the term “sickle cell disease” refers to a variety of clinical problems attendant upon sickle cell anemia, especially in those subjects who are homozygotes for the sickle cell substitution in HbS. Among the constitutional manifestations referred to herein by use of the term of sickle cell disease are delay of growth and development, an increased tendency to develop serious infections, particularly due to pneumococcus, marked impairment of splenic function, preventing effective clearance of circulating bacteria, with recurrent infarcts and eventual destruction of splenic tissue. Also included in the term “sickle cell disease” are acute episodes of musculoskeletal pain, which affect primarily the lumbar spine, abdomen, and femoral shaft, and which are similar in mechanism and in severity to the bends. In adults, such attacks commonly manifest as mild or moderate bouts of short duration every few weeks or months interspersed with agonizing attacks lasting 5 to 7 days that strike on average about once a year. Among events known to trigger such crises are acidosis, hypoxia and dehydration, all of which potentiate intracellular polymerization of HbS (J. H. Jandl, Blood: Textbook of Hematology, 2nd Ed., Little, Brown and Company, Boston, 1996, pages 544-545). As used herein, the term “thalassemia” encompasses hereditary anemias that occur due to mutations affecting the synthesis of hemoglobin. Thus, the term includes any symptomatic anemia resulting from thalassemic conditions such as severe or .beta.-thalassemia, thalassemia major, thalassemia intermedia, .alpha.-thalassemias such as hemoglobin H disease.

As used herein, “thalassemia” refers to a hereditary disorder characterized by defective production of hemoglobin. Examples of thalassemias include β and α thalassemia. β thalassemias are caused by a mutation in the beta globin chain, and can occur in a major or minor form. In the major form of β thalassemia, children are normal at birth, but develop anemia during the first year of life. The mild form of β thalassemia produces small red blood cells a thalassemias are caused by deletion of a gene or genes from the globin chain.

As used herein, “antisickling proteins” include proteins which prevent or reverse the pathological events leading to sickling of erythrocytes in sickle cell conditions. In one embodiment of the invention, the transduced cells of the invention are used to deliver antisickling proteins to a subject with a hemoglobinopathic condition. Antisickling proteins also include mutated β-globin genes comprising antisickling amino acid residues.

As used herein, the term “insulator” or “insulator element,” used interchangeably herein, refers to an exogenous DNA sequence that can be added to a vector of the invention to prevent, upon integration of the vector into a host genome, nearby genomic sequences from influencing expression of the integrated trans-gene(s). Conversely, the insulator element prevents the integrated vector from influencing expression of nearby genomic sequences. This is generally achieved as the insulator is duplicated upon integration of the vector into the genome, such that the insulator flanks the integrated vector (e.g., within the LTR region) and acts to “insulate” the integrated DNA sequence. Suitable insulators for use in the invention include, but are not limited to, the chicken β-Globin insulator (see Chung et al. Cell (1993) 74:505; Chung et al., PNAS (1997) 94:575; and Bell et al. Cell 1999 98:387, incorporated by reference herein). Examples of insulator elements include, but are not limited to, an insulator from an β-globin locus, such as chicken HS4.

As used herein, unless the context makes clear otherwise, “treatment,” and similar words such as “treated,” “treating” etc., indicates an approach for obtaining beneficial or desired results, including and preferably clinical results. Treatment can involve optionally either the reduction or amelioration of symptoms of the disease or condition, or the delaying of the progression of the disease or condition.

As used herein, unless the context makes clear otherwise, “prevent,” and similar words such as “prevented,” “preventing” etc., indicates an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

As used herein, an “effective amount” or a “therapeutically effective amount” of an agent or a substance is that amount sufficient to affect a desired biological effect, such as beneficial results, including clinical results.

As used herein “pharmaceutically acceptable carrier” includes any and all solvents, dispersion media, coatings, antibacterial and antifungal agents, isotonic and absorption delaying agents, and the like that are physiologically compatible. In one embodiment, the carrier is suitable for parenteral administration. Preferably, the carrier is suitable for administration directly into an affected joint. The carrier can be suitable for intravenous, intraperitoneal or intramuscular administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the transduced cells, use thereof in the pharmaceutical compositions of the invention is contemplated.

In the following description, certain specific details are set forth in order to provide a thorough understanding of various embodiments of the invention. However, one skilled in the art will understand that the invention may be practiced without these details.

Unless the context requires otherwise, throughout the present specification and claims, the word “comprise” and variations thereof, such as, “comprises” and “comprising” are to be construed in an open, inclusive sense, that is as “including, but not limited to.” As used herein, the terms “include” and “comprise” are used synonymously.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polypeptide or polynucleotide length, are to be understood to include any integer within the recited range, unless otherwise indicated.

As used herein, “about” means±20% of the indicated range, value, or structure, unless otherwise indicated. It should be understood that the terms “a” and “an” as used herein refer to “one or more” of the enumerated components.

The use of the alternative (e.g., “or”) should be understood to mean either one, both, or any combination thereof of the alternatives. As used herein, the terms “include” and “comprise” are used synonymously.

In addition, it should be understood that the individual vectors, or groups of vectors, derived from the various combinations of the structures and substituents described herein, are disclosed by the present application to the same extent as if each vector or group of vectors was set forth individually. Thus, selection of particular vector structures or particular substituents is within the scope of the present disclosure.

Methods of Producing and Selecting Transduced Cells, and Related Methods of Enhancing the Reconstitution of Cell Populations in a Transplant Recipient by Transduced Cells and Delivering a Therapeutic Polypeptide to a Subject in Need Thereof

Certain aspects of the current invention arise from the unexpected finding that puromycin-based selection systems can be effective in selecting transduced multipotent cell, including transduced stem cells, while maintaining a sufficient degree of multipotent cell quality and engraftment capability. A key aspect of this finding is the identification of appropriate puromycin concentrations and appropriate lengths of time to expose the cells to puromycin, such that untransduced cells are depleted, while transduced multipotent cells maintain their multipotency and engraftment capability. For example, in certain instances, puromycin-selected transduced hematopoietic stem cells selected according to methods of the present invention are capable of reconstituting the hematopoietic cells of a transplant recipient in whom such cells are transplanted. This reconstitution may be long-term reconstitution.

Accordingly, the present invention provides novel methods of selecting transduced multipotent cells, including stem cells, as well as related methods of using puromycin selection in producing transduced multipotent cells and cell populations enriched in transduced multipotent cells. While the description and examples provided herein focus on the transduction and selection of multipotent cells, including hematopoietic stem cells in particular, the methods and transfer vectors of the instant invention may also be used to transduce and select other cell types, including other types of multipotent or stem cells and fragile cells previously not amenable to selection of transduced cells for therapeutic uses. Such cell may include, but are not limited to, embryonic stem cells, induced pluripotent stem cells and somatic stem cells, including hematopoietic stem cells, adipose tissue derived stem cells, and umbilical cord matrix stem cells. Cell used according to the methods of the present invention may be obtained from any animal, preferably a mammal, and more preferably a human, and they may be transplanted into any animal, preferably a mammal, and more preferably a human.

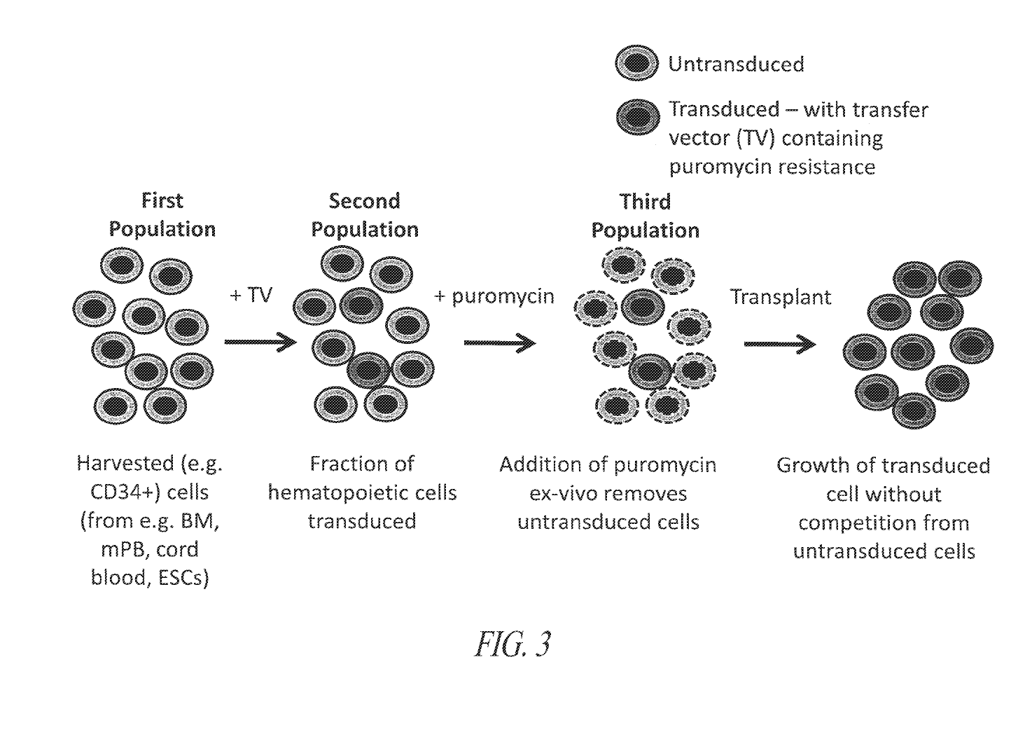

FIG. 3 provides a schematic diagram depicting one process of the present invention for selecting transduced cell prior to transplantation into a recipient. These transduced cells are transplanted into a recipient, where the transduced multipotent cells grow without competition from untransduced cells and eventually reconstitute a cell population within the recipient.

In one embodiment, the present invention provides a method of selecting transduced multipotent cells, which may include stem cells, comprising contacting a first population of cells comprising multipotent cells, which may include stem cells, with 1-25 μg/ml of puromycin for four days or less, wherein said first population of cells was previously contacted with a transfer vector comprising a polynucleotide sequence encoding a puromycin resistance polypeptide operably linked to a promoter sequence, thereby producing a second population of cells comprising transduced multipotent cells, which may include transduced stem cells. In particular embodiments, the first population of cells is contacted with the transfer vector under conditions and for a time sufficient to permit transduction of the cells by the transfer vector or integration of the polynucleotide sequence into the genome of cells.

The above method of selecting transduced cells may be used in the context of producing transduced multipotent cells, which may include transduced stem cells. Accordingly, in a related embodiment, the present invention provides a method of producing transduced multipotent cells, comprising: (i) contacting a first population of cells comprising multipotent cells, which may include stem cells, with a transfer vector comprising a polynucleotide sequence encoding a puromycin resistance polypeptide operably linked to a promoter sequence, thereby producing a second population of cells comprising multipotent cells, which may include stem cells, comprising said transfer vector; and (ii) contacting the second population of cells with 1-25 μg/ml puromycin for 4 days or less, thereby producing a third population of cells, wherein said third population of cells comprises a higher percentage of transduced multipotent cells than said second population of cells. In particular embodiments, the first population of cells is contacted with the transfer vector under conditions and for a time sufficient to permit transduction of the cells by the transfer vector or integration of the polynucleotide sequence into the genome of cells.

In particular embodiments of any of the puromycin selection methods of the present invention, at least 50%, at least 55%, at least 60%, at least 65, at least 70%, at least 75%, at least 80%, at least 85%, or at least 90% of the cells remaining following puromycin selection (e.g., the third population) are transduced. In certain embodiments, at least 75% of the cells in the third population are transduced.

The methods described above provide a cell population comprising transduced multipotent cells, including transduced stem cells, which may be used to reconstitute a cell population within a transplant recipient. Thus, in particular embodiments, the present invention includes a method of enhancing the reconstitution by transduced stem cells of a cell population within a subject, comprising producing transduced stem cells as described above and transplanting a plurality of the third population of cells into said subject.

In particular embodiments, the transduced multipotent cells within the third population of cells are capable of producing at least two distinct cell lineages containing the transfer vector for at least four months, at least six months, or at least twelve months following introduction of the third population of cells into a subject, although not necessarily immediately following introduction of the cells into the subject. It is recognized that the production of the at least two distinct cell lineages may not be immediate, i.e., a lag period may exist; however, in particular embodiments, the at least four month, at least six month, or at least twelve month time period begins within the 24 months, 28 months, 36 months, or 48 months immediately following introduction of said third population of cells into the living subject.

Transduced cells produced according to methods of the present invention may be used therapeutically, e.g., they may be implanted into a subject in need thereof. For example, transduced cells that express a therapeutic polypeptide may be transplanted into a recipient subject who expresses a reduced amount of the therapeutic polypeptide or expresses a mutant form of the therapeutic polypeptide. Therefore, in certain instances, cells to be transduced are obtained from a subject in need of transplantation by the transduced stem cells (autologous transplant). In other instances, the cells to be transduced are obtained from another donor, who may be tissue matched to a subject in need of transplantation by the transduced stem cells (allogenic transplant).

Cells may be obtained from a variety of different sources in a donor, using methods known and available in the art. For instance, hematopoietic cells, including hematopoietic stem cells (HSC), may be obtained from bone marrow using a needle, peripheral blood cells may be obtained by apheresis, and cells may be filtered from blood in the umbilical cord after a child is born. Cells may be purified from other tissue components such as fat and extracellular matrix using conventional techniques to produce a cell population, which may contain stem cells.

Multipotent cells, including stem cells, may be selected from or enriched within a cell population prior to transduction. For example, stem cells may be selected based upon their expression of at least one marker associated with stem cells or by physical separation means. Examples of markers associated with stem cells include CD34, Thy-1 and rho. Cells expressing these markers may be purified from or enriched within a cell population by a variety of means, including fluorescence activated cell sorting (FACS) using antibodies specific for one or more marker. In particular embodiments, a cell population obtained from a donor is enriched for CD34⁺ cells, or CD34⁺ cells are purified from other cells, before transduction.

Transduction of cells is performed using a transfer vector capable of expressing a puromycin resistance polypeptide, including any of the transfer vectors described infra. Thus, the transfer vector comprises a polynucleotide sequence that encodes a puromycin resistance polypeptide, which may be any polypeptide that confers resistance to puromycin in cells expressing it. The pac gene encoding a Puromycin N-acetyl-transferase (PAC) has been isolated from a Streptomyces producing strain (de la Luna, S. & Ortin, J. (1992). Methods Enzymol. 216:376-385; de la Luna, S., et al. (1988). Gene 62:121-126). It is located in a region of the pur cluster linked to the other genes determining the puromycin biosynthetic pathway. The expression of pac gene confers puromycin resistance to transfected mammalians cells expressing it. However, exogenous DNA, such as puromycin resistance genes from bacterial origin, may be poorly suitable for expression in mammalian cells. First, codon usage in bacteria is very different from mammalian codon usage. In addition, the foreign (bacterial) DNA composition in CpG dinucleotides is very different from the CpG distribution in mammalian DNA. This difference elicits two phenomena which negatively affect gene expression: recognition of the bacterial DNA as foreign by the mammalian immune system and methylation on the cytosine residue of CpG, leading to gene silencing. Therefore, to avoid pac gene silencing in transfer vectors, modified pac genes may be used in transfer vectors according to the present invention. In certain embodiments, modified pac genes are codon-optimized for mammalian expression and/or some or all CpG motifs have been removed. In particular embodiments, the polynucleotide sequence that encodes a puromycin resistance polypeptide contains only the coding regions of a pac gene or modified pac gene. In certain embodiments, the polynucleotide sequence that encodes a puromycin resistance polypeptide has the nucleic acid sequence set forth in SEQ ID NO:1. In certain embodiments, the puromycin resistance polypeptide has the amino acid sequence set forth in SEQ ID NO:2. The present invention also contemplates the use of functional fragments or variants of any of these puromycin resistance polypeptides.

In particular embodiments, the transfer vector that expresses a puromycin resistance polypeptide is a retroviral vector, e.g., a lentiviral vector, such as an HIV vector. Lentiviral infection has several advantages over other transduction methods, including high-efficiency infection of dividing and non-dividing cells, long-term stable expression of a transgenic, and low immunogenicity. Various transfer vectors that may be used according to the present invention are described supra.

The production of infectious viral particles and viral stock solutions may be carried out using conventional techniques. Methods of preparing viral stock solutions are known in the art and are illustrated by, e.g., Y. Soneoka et al. (1995) Nucl. Acids Res. 23:628-633, and N. R. Landau et al. (1992) J. Virol. 66:5110-5113. For example, viral particles may be produced using either a packaging cell line or by transient transfection of a transfer vector in combination with plasmids that produce viral proteins used in packaging and production of infectious viral particles. Examples of suitable packaging cell lines are described, e.g., in U.S. Pat. Nos. 6,958,226, 6,620,595, 5,739,018, 5,686,279 and 5,591,624. In particular embodiments, HIV type 1 (HIV-1) based viral particles may be generated by co-expressing the virion packaging elements and the transfer vector in a producer cell. These cells may be transiently transfected with a number of plasmids. Typically from three to four plasmids are employed, but the number may be greater depending upon the degree to which the lentiviral components are broken up into separate units. For example, one plasmid may encode the core and enzymatic components of the virion, derived from HIV-1. This plasmid is termed the packaging plasmid. Another plasmid typically encodes the envelope protein(s), most commonly the G protein of vesicular stomatitis virus (VSV G) because of its high stability and broad tropism. This plasmid may be termed the envelope expression plasmid. Yet another plasmid encodes the genome to be transferred to the target cell, that is, the vector itself, and is called the transfer vector. The packaging plasmids can be introduced into human cell lines by known techniques, including calcium phosphate transfection, lipofection or electroporation, generally together with a dominant selectable marker, such as neomycin, DHFR, Glutamine synthetase or ADA, followed by selection in the presence of the appropriate drug and isolation of clones. The selectable marker gene can be linked physically to the packaging genes in the construct. Recombinant viruses with titers of several millions of transducing units per milliliter (TU/ml) can be generated by this technique and variants thereof. After ultracentrifugation concentrated stocks of approximately 10⁹ TU/ml can be obtained.

In one exemplary method of producing a stock solution of lentivirus according to the present invention, lentiviral-permissive cells (referred to herein as producer cells) are transfected with the transfer vector and other vectors that express viral proteins (or derivatives thereof) necessary for the production of viral particles. The cells are then grown under suitable cell culture conditions, and the lentiviral particles collected from either the cells themselves or from the cell media as described above. Suitable producer cell lines include, but are not limited to, the human embryonic kidney cell lines 293 and 293T, the equine dermis cell line NBL-6, and the canine fetal thymus cell line Cf2TH. Examples of such multi-plasmid viral packaging systems are described in U.S. Pat. Nos. 5,994,136, 6,924,144, 7,250,299, 6,790,641, and 6,013,516.

Infectious virus particles may be collected from the packaging cells using conventional techniques. For example, the infectious particles can be collected by cell lysis, or collection of the supernatant of the cell culture, as is known in the art. Optionally, the collected virus particles may be purified if desired. Suitable purification techniques are well known to those skilled in the art.

Other methods relating to the use of viral vectors in gene therapy, which may be utilized according to certain embodiments of the present invention, can be found in, e.g., Kay, M. A. (1997) Chest 111(6 Supp.):1385-1425; Ferry, N. and Heard, J. M. (1998) Hum. Gene Ther. 9:1975-81; Shiratory, Y. et al. (1999) Liver 19:265-74; Oka, K. et al. (2000) Curr. Opin. Lipidol. 11:179-86; Thule, P. M. and Liu, J. M. (2000) Gene Ther. 7:1744-52; Yang, N. S. (1992) Crit. Rev. Biotechnol. 12:335-56; Alt, M. (1995) J. Hepatol. 23:746-58; Brody, S. L. and Crystal, R. G. (1994) Ann. N.Y. Acad. Sci. 716:90-101; Strayer, D. S. (1999) Expert Opin. Investig. Drugs 8:2159-2172; Smith-Arica, J. R. and Bartlett, J. S. (2001) Curr. Cardiol. Rep. 3:43-49; and Lee, H. C. et al. (2000) Nature 408:483-8.

Viruses may be used to infect cells ex vivo or in vitro using standard transfection techniques well known in the art For example, when cells, for instance CD34⁺ cells, dendritic cells, peripheral blood cells or tumor cells are transduced ex vivo, the vector particles may be incubated with the cells using a dose generally in the order of between 1 to 50 multiplicities of infection (MOI) which also corresponds to 1×10⁵ to 50×10⁵ transducing units of the viral vector per 10⁵ cells. This, of course, includes amount of vector corresponding to 1, 2, 3, 4, 5, 6, 7, 8, 9, 10, 15, 20, 25, 30, 35, 40, 45, and 50 MOI. Typically, the amount of vector may be expressed in terms of HEK293, HEK293T, NIH3T3 or HeLa transducing units (TU).

Once cells have been infected with virus, cells transduced by the transfer vector and expressing the puromycin resistance gene are selected by contacting the cells with puromycin or a functional fragment or derivative thereof. Puromycin is commercial available, e.g., from Clontech (Mountain View, Calif.). In particular embodiments, cells are contacted with puromycin for five days or less, four days or less, three days or less, 2 days or less, or one day or less. In particular embodiments, cells are contacted with puromycin for 12-24 hours, 12-36 hours, 12-48 hours, or for 24-48 hours. Typically, this indicates the number of consecutive hours or days of continued exposure of the cells to puromycin. In particular embodiments, cells are contacted with puromycin at a concentration in the range of 1-25 μg/ml, 1-20 μg/ml, 1-10 μg/ml, or at a puromycin concentration of about 2 μg/ml, about 3 μg/ml, about 4 μg/ml, about 5 μg/ml, about 6 μg/ml, about 7 μg/ml, about 8 μg/ml, about 9 μg/ml or about about 10 μg/ml. As demonstrated in the accompanying Examples, treatment with as little as 5 μg/ml of puromycin for as short a time as 24 hours resulted in a significant selection and enrichment of transduced cells.

Prior to, during, and/or following puromycin selection, the cells may be cultured in media suitable for the maintenance, growth, or proliferation of the cells. Suitable culture media and conditions are well known in the art. Following puromycin selection, the selected cells may be cultured under conditions suitable for their maintenance, growth or proliferation. In particular embodiments, the selected cells are cultured for about 7 to about 14 days before transplantation.

Puromycin selected cells may be assayed to determine whether they have been successfully transduced with the transfer vector. In certain embodiments, the presence of the transfer vector is determined by polymerase chain reaction (PCR) using primers that specifically amplify a region of the transfer vector not present in untransduced cells. For example, PCR analysis may be performed on individual colonies, or on clonal cell populations. In particular embodiments, puromycin selection results in the enrichment of transduced cells within the resulting selected cell population. In certain embodiments, at least 50%, at least 60%, at least 70%, at least 80%, at least 90% or 100% of the cells are transduced with the transfer vector. In particular embodiments, this represents an at least two-fold, at least three-fold, at least four-fold, or at least five-fold enrichment of transduced cells.

During or following puromycin selection of transduced cells, the selected cells may be cultured under conditions that promote the expansion of stem cells or multipotent cells. Any method known in the art may be used. In certain embodiments, during or following selection, the cells are cultured in the presence of one or more small molecules that promote the expansion of stem cells or multipotent cells. Examples of such molecules include, but are not limited to, SR1, which antagonizes the aryl hydrocarbon receptor, and valproic acid. In other embodiments, during or following selection, the cells are cultured in the presence of one or more growth factors that promote the expansion of stem cells or multipotent cells. Examples of growth factors that promote the expansion of stem cells or multipotent cells include, but are not limited to, fetal liver tyrosine kinase (Flt3) ligand, stem cell factor, and interleukins 6 and 11, which have been demonstrated to promote self-renewal of murine hematopoietic stem cells. Others include Sonic hedgehog,’ which induces the proliferation of primitive hematopoietic progenitors by activation of bone morphogenetic protein 4, Wnt3a, which stimulates self-renewal of HSCs, brain derived neurotrophic factor (BDNF), epidermal growth factor (EGF), fibroblast growth factor (FGF), ciliary neurotrophic factor (CNF), transforming growth factor-β (TGF-β), a fibroblast growth factor (FGF, e.g., basic FGF, acidic FGF, FGF-17, FGF-4, FGF-5, FGF-6, FGF-8b, FGF-8c, FGF-9), granulocyte colony stimulating factor (GCSF), a platelet derived growth factor (PDGF, e.g., PDGFAA, PDGFAB, PDGFBB), granulocyte macrophage colony stimulating factor (GMCSF), stem cell factor (SCF), stromal cell derived factor (SCDF), insulin like growth factor (IGF), thrombopoietin (TPO) or interleukin-3 (IL-3). In particular embodiments, during or following selection, the cells are cultured in the presence of both one or more small molecules and one or more growth factors that promote expansion of stem cells or multipotent cells.

In certain situations, it may be desirable to express the puromycin resistance polypeptide only during a limited time period, e.g., during the puromycin selection process. Therefore, in certain embodiments, the polynucleotide that encodes the puromycin resistance polypeptide is operably linked to a transiently inducible promoter, so that expression of the puromycin resistance polypeptide can be turned on post-transduction, e.g., before and/or during contact of the cells with puromycin, and then subsequently turned off following selection of cells expressing the puromycin resistance polypeptide. Thus, the puromycin resistance polypeptide would not be expressed in transplant recipients and this should reduce or mitigate possible undesired effects of its expression, such as the activation of endogenous oncogenes within the cells or immune reaction against the puromycin resistance polypeptide and associated rejection by the transplant recipient before the establishment of immune tolerance.

A variety transiently inducible promoter systems are known and available in the art, including, e.g., the Cre/loxP system, two tetracycline-responsive Tet systems (Tet-On, Tet-Off), the glucocorticoid-responsive mouse mammary tumor virus promoter (MMTVprom), the ecdysone-inducible promoter (EcP), and the T7 promoter/T7 RNA polymerase system (T7P). Any of these may be used to drive the inducible expression of the puromycin resistance gene according to certain embodiments of the present invention.

In another embodiment, the polynucleotide that encodes the puromycin resistance polypeptide is operably linked to a promoter that is more active in stem cells or multipotent cells as compared to its activity in differentiated cells, so that expression of the puromycin resistance polypeptide occurs in transduced stem cells and multipotent cells, thus facilitating puromycin selection, but following transplant of the transduced cells into a recipient, expression of the puromycin resistance polypeptide is reduced in differentiated cells generated from the implanted transduced stem cells and multipotent cells. In particular embodiments, the promoter is more active in hematopoietic stem cells than it is in red blood cells. A variety of promoters having greater activity in multipotent cells, e.g., stem cells, as compared to differentiated cells are known and available in the art.

As described above, transduced cells produced according to methods of the present invention may be used to deliver a therapeutic polypeptide to a subject in need thereof. Accordingly, the transfer vector may comprise both a polynucleotide sequence encoding the puromycin resistance polypeptide and polynucleotide sequence encoding a therapeutic polypeptide. In one embodiment, each of these polynucleotide sequences is operably linked to the same promoter, but in other embodiments, each of these polynucleotide sequences is operably linked to a different promoter, such that expression of the puromycin resistance gene and expression of the therapeutic polypeptide are regulated independently. In particular embodiments, one or both promoters are constitutive promoters, inducible promoters, or tissue specific promoters. In certain embodiments, the promoter driving expression of the puromycin resistance gene is an inducible promoter, as discussed above. In certain embodiments, the promoter driving expression of the therapeutic polypeptide has reduced activity in stem cells or multipotent cells as compared to its activity in at least cell type differentiated from such stem cells or multipotent cells. Accordingly, expression of the therapeutic polypeptide can be enhanced in or limited to one or more differentiated cell types. In particular embodiments, the promoter driving expression of the therapeutic polypeptide is active in one or more differentiated hematopoietic cells, such as, e.g., red blood cells.

Tissue-specific promoters that preferentially drive expression in one or more differentiated tissue are known and available in the art and include, e.g., the human β-globin promoter. Tissue specificity may be further enhanced by including a tissue specific enhancer element. For example, an enhancer element upstream of the mouse Gata1 IE (1st exon erythroid) promoter, mHS-3.5, can direct both erythroid and megakaryocytic expression.

The present invention further contemplates the inclusion of a suicide gene in the transfer vector, so that transduced cells may be negatively selected if desired. In particular embodiments, the suicide gene is operably linked to an inducible promoter. In various embodiments, it is operably linked to a constitutive promoter. For example, the suicide gene may be constitutively active and its expression induced when desired using an operatively linked inducible promoter. As further example, the suicide gene may be inducibly active and either constitutively expressed or inducibly expressed using an operatively linked constitutive promoter or inducible promoter, respectively.

In particular embodiments, methods of the present invention utilize the expression of both a puromycin resistance polypeptide and a conditional suicide gene, such as herpes simplex virus-thymidine kinase. Thus, neoplastic cells that may be generated upon vector-mediated insertional mutagenesis in the transplant recipient would be rendered treatable by chemically-induced cell suicide (e.g., ganciclovir). Other conditional suicide genes may be used in lieu of herpes simplex virus-thymidine kinase encoding gene. In particular embodiments, the puromycin resistance and the conditional cell suicide proteins may be co-expressed by means of (i) an internal ribosomal entry site (IRES) within a polycistronic vector, (ii) by cleavage of a precursor protein or (iii) as a fusion protein.

Constitutive promoters for expression in mammalian cells are also known and available in the art and include, e.g., the phosphoglycerate kinase (PGK) promoter and derivatives thereof (e.g., the mouse PGK promoter), the simian virus 40 early promoter (SV40), the cytomegalovirus immediate-early promoter (CMV), human Ubiquitin C promoter (UBC), human elongation factor 1a promoter (EFTA), and chicken β-Actin promoter coupled with CMV early enhancer (CAGG).

As discussed above, methods of the present invention may be used to produce transduced cells for the delivery of a therapeutic polypeptide to a subject in need thereof. In particular embodiments, these methods are practiced to provide a therapeutic polypeptide to one or more one or more cell types capable of being differentiated from the transduced stem cells or multipotent cells. In certain embodiments, the one or more cell type is a hematopoietic cell type, which includes myeloid (monocytes and macrophages, neutrophils, basophils, eosinophils, erythrocytes, megakaryocytes/platelets, dendritic cells), and lymphoid lineages (T-cells, B-cells, NK-cells).

In particular embodiments, transduced cells produced according to methods of the present invention are used to treat a disease or disorder of the hematopoietic system, such as a hemoglobinopathy, anemia or thalassemia. As used herein, the term “hemoglobinopathy” or “hemoglobinopathic condition” includes any disorder involving the presence of an abnormal hemoglobin molecule in the blood. Examples of hemoglobinopathies included, but are not limited to, hemoglobin C disease, hemoglobin sickle cell disease (SCD), sickle cell anemia, and thalassemias. Also included are hemoglobinopathies in which a combination of abnormal hemoglobins are present in the blood (e.g., sickle cell/Hb-C disease).

The term “sickle cell anemia” or “sickle cell disease” is defined herein to include any symptomatic anemic condition which results from sickling of red blood cells. Manifestations of sickle cell disease include: anemia; pain; and/or organ dysfunction, such as renal failure, retinopathy, acute-chest syndrome, ischemia, priapism and stroke. As used herein the term “sickle cell disease” refers to a variety of clinical problems attendant upon sickle cell anemia, especially in those subjects who are homozygotes for the sickle cell substitution in HbS. Among the constitutional manifestations referred to herein by use of the term of sickle cell disease are delay of growth and development, an increased tendency to develop serious infections, particularly due to pneumococcus, marked impairment of splenic function, preventing effective clearance of circulating bacteria, with recurrent infarcts and eventual destruction of splenic tissue. Also included in the term “sickle cell disease” are acute episodes of musculoskeletal pain, which affect primarily the lumbar spine, abdomen, and femoral shaft, and which are similar in mechanism and in severity to the bends. In adults, such attacks commonly manifest as mild or moderate bouts of short duration every few weeks or months interspersed with agonizing attacks lasting 5 to 7 days that strike on average about once a year. Among events known to trigger such crises are acidosis, hypoxia and dehydration, all of which potentiate intracellular polymerization of HbS (J. H. Jandl, Blood: Textbook of Hematology, 2nd Ed., Little, Brown and Company, Boston, 1996, pages 544-545).

As used herein, “thalassemia” refers to a hereditary disorder characterized by defective production of hemoglobin. Examples of thalassemias include β and α thalassemia. β thalassemias are caused by a mutation in the beta globin chain, and can occur in a major or minor form. In the major form of β thalassemia, children are normal at birth, but develop anemia during the first year of life. The mild form of β thalassemia produces small red blood cells a thalassemias are caused by deletion of a gene or genes from the globin chain. Thus, the term includes any symptomatic anemia resulting from thalassemic conditions such as severe or β-thalassemia, thalassemia major, thalassemia intermedia, α-thalassemias such as hemoglobin H disease.

In particular embodiments, the therapeutic polypeptide is an antisickling protein. As used herein, “antisickling proteins” include proteins which prevent or reverse the pathological events leading to sickling of erythrocytes in sickle cell conditions. In one embodiment of the invention, the transduced cell of the invention is used to deliver antisickling proteins to a subject with a hemoglobinopathic condition. Antisickling proteins also include polypeptides expressed from mutated β-globin genes comprising antisickling amino acid residues, e.g., a mutated β-globin having a substitution of threonine at position 87 with glutamine. A gene or cDNA sequence encoding a therapeutic polypeptide can be obtained for insertion into the transfer vector through a variety of techniques known to one of ordinary skill in the art.

The present invention further includes pharmaceutical compositions comprising transduced cells produced according to methods described herein and a pharmaceutically acceptable carrier. In one embodiment, the carrier is suitable for parenteral administration. The carrier can be suitable for intravenous, intraperitoneal or intramuscular administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art.

Methods of Reducing Myelosuppression in Transplant Recipients

Another aspect of the present invention relates to the inhibition, prevention, or amelioration of the transient myelosuppression that can occur in transplant recipients, particularly those patients who received myeloablative treatment prior to transplant of transduced stem cells or multipotent cells, e.g., transduced hematopoietic stem cells or multipotent cells. This aspect of the present invention is particularly relevant when the transduced cells have been selected according to a methods of the present invention prior to transplantation, due to the loss of untransduced cells, e.g., hematopoietic cells, from prior ex-vivo puromycin treatment. When fewer cells are transplanted, there is a higher risk of myelosuppression. In addition, there can be a substantial time delay before stem cells effect substantial repopulation.

Accordingly, the present invention provides methods to inhibit transient myelosuppression by co-transplanting into a subject the population of puromycin-selected transduced cells (which includes multipotent cells) with a separate population of cells capable of producing hematopoietic cells that are depleted in a subject suffering from myelosuppression, such as red blood cells, white blood cells, and/or platelets. In particular embodiments, this separate population of cells does not include stem cells capable of self-renewal or long-term repopulation of the subject or includes only a small amount of such cells, e.g., less than 10%, less than 5%, less than 1%, less than 0.1%, or less than 0.01%. Thus, the separate population of cells provides transient or short term repopulation of hematopoietic cells, thereby inhibiting or reducing myelosuppression, while transduced multipotent cells, including transduced stem cells, present in the puromycin-selected transduced cells provide long-term repopulation of hematopoietic cells in the subject. In certain embodiments, the population of cells included to provide short term repopulation will include a lower percentage of stem cells that the population of cells comprising transduced stem cells, which is used to provide long term repopulation. However, it is understood that one or both of these different cell populations may include one or more types of cells selected from: stem cells, multipotent cells, progenitor cells, and differentiated cells. Steps may be taken, however, to reduce the number of stem cells present in the population of cells being used for short term repopulation, including those described herein.

According to various embodiments of methods of the present invention, the cells used to provide short term repopulation may be either transduced or non-transduced cells. For example, in certain embodiments, the cells used to provide short term repopulation are transduced and selected as described above, but are then expanded and/or differentiated as described herein.

In particular embodiments, short term repopulation or transient repopulation of a cell lineage within a transplant patient indicates a duration of repopulation of at least one month, at least two months, at least three months, or at least four months. In particular embodiments, short term repopulation or transient repopulation of a cell lineage within a transplant patient indicates a duration of repopulation of less than one year, less than six months or less than four months. In particular embodiments, short term repopulation or transient repopulation of a cell lineage within a transplant patient indicates a duration of repopulation of between one month and one year, between one month and six months, or between one month and four months.

In particular embodiments, long-term repopulation of a cell lineage within a transplant patient indicates a duration of repopulation of at least four months, which may occur at any time following transplantation. In particular embodiments, long term repopulation of a cell lineage within a transplant patient indicates a duration of repopulation of more than one year, six months or four months. The time period during which said repopulation occurs may be at any time following transplantation. In certain embodiments, it begins within one year, 18 months or two years following transplantation.

Thus, in one embodiment, the present invention includes a method of inhibiting myelosuppression following transplantation of transduced stem cells into a transplant recipient, comprising transplanting a first population of cells comprising transduced stem cells into a transplant recipient in combination with a second population of cells having a reduced percentage of stem cells as compared to the first population of cells. In particular embodiments, the transplant recipient has undergone a myeloablative regimen prior to transplantation, and, in particular embodiments, the transplant recipient has a reduced number of hematopoietic precursor cells capable of differentiating into various differentiated hematopoietic cells, such as red blood cells. In certain embodiments, the first population of cells comprises stem cells transduced with a transfer vector comprising a polynucleotide encoding a therapeutic polypeptide. In particular embodiments, the first population of cells is capable of long term repopulation of hematopoietic cells within the transplant recipient, and the second population of cells is capable of short term repopulation of hematopoietic cells within the transplant recipient. In particular embodiments, the first population of cells comprises transduced stem cells. In various embodiments, the second population of cells comprises transduced and/or non-transduced progenitor cells.

In various embodiments, the second population of cells is obtained from the transplant recipient or from another donor (prior to any subsequent culturing or modification). Thus, the first population of cells may be obtained from the same or a different initial source than the second population of cells. For example, the first population of cells may be produced using cells obtained from the transplant recipient, whereas the second population of cells is prepared from cells obtained from an allogeneic donor. In another example, both the first population of cells and the second population of cells are produced from cells obtained from the transplant recipient (at the same time or at different times).

Given that the cells present in the second population of cells are transplanted into a recipient to provide transient repopulation of a cellular compartment, such as the hematopoietic system, certain embodiments of the present invention contemplate that the second population of cells will have a lower percentage of stem cells than the first population of cells. Therefore, long-term engraftment and repopulation will be performed by the puromycin-selected transduced stem cells present in the first population of cells. Thus, in particular embodiments, the second population of cells is cultured under conditions whereby the long-term repopulating stem cell population is depleted, while maintaining or expanding transient repopulating hematopoietic progenitor populations by e.g. certain growth factor combinations. Accordingly, in particular embodiments, methods of this aspect of the present invention include contacting the second population of cells with an agent that either depletes or removes stem cells from the population, an agent that inhibits stem cell growth or proliferation, or an agent that induces or promotes differentiation of stem cells into progenitor cells. In specific embodiments, the stem cell is a hematopoietic stem cell.

Agents that may be used for depleting or removing stem cells from a population include, but are not limited to, antibodies specific for cell surface markers expressed on stem cells (e.g., CD34, Sca1, Lin, c-kit), which may be used to bind and remove or enrich stem cells from a cell population. Agents that may be used to inhibit stem cell growth or proliferation include any known in the art.

Agents that may be used to induce or promote differentiation of a multipotent stem, e.g., a stem cell into a progenitor cell include, e.g., various cytokines and growth factors, and combinations thereof. Examples of cytokines that may be used for such ex vivo expansion or differentiation purposes include, but are not limited to, IL-1 (i.e., IL-1β), IL-3, IL-6, IL-11, G-CSF, GM-CSF, and analogs thereof. Suitable growth factors for ex vivo expansion purposes may be selected from c-kit ligand (SCF or SF), FLT-3 ligand (FL), thrombopoietin (TPO), erythropoietin (EPO), and analogs thereof. As used herein, analogs include variants of the cytokines and growth factors having the characteristic biological activity of the naturally occurring forms. In one embodiment, the cytokine and growth factor mixture in its base composition has stem cell factor (SCF), FLT-3 ligand (FL), and thromobopoietin (TPO). In other embodiments, the cytokine and growth factor mixture has an additional cytokine selected from IL-3, IL-6, IL-11, G-CSF, GM-CSF, and combinations thereof, and particularly from IL-3, IL-6, IL-11, and combinations thereof. Thus, in one embodiment, the cytokine and growth factor mixture has the composition SCF, FL, TPO, and IL-3 while in another embodiment, the mixture has the composition SCF, FL, TPO, and IL-6. One combination of the additional cytokine is IL-6 and IL-11 such that the cytokine and growth factor mixture has the composition SCF, FL, TPO, IL-6 and IL-11. Methods of culturing hematopoietic stem cells in a culture medium comprising a cytokine and growth factor mixture to expand the myeloid progenitor cells are also described in U.S. Patent Application Publication No. 2006/0134783. This application describes expansion of the myeloid population of progenitor cells using CD34⁺, CD90⁺ HSCs as a starting population. The cells are treated with a combination of KITL, FLT3L, TPO, and IL-3, optionally in combination with IL-6, which seems to have a synergistic effect on the myeloid expansion. Particular combinations include KITL, FLT3L, TPO, optionally with IL-3, further optionally with a combination of IL3, IL-6, and IL-11.

In particular embodiments, the second population of cells comprises progenitor or precursor cells, which are relatively immature cells that are precursors to a fully differentiated cell of the same tissue type. Progenitor or precursor cells, e.g., hematopoietic progenitor cells, are capable of proliferating, but they have a limited capacity to differentiate into more than one cell type. In addition, progenitor or precursor cells lack the capacity for long-term self-renewal. In particular embodiments, hematopoietic progenitor cells may restore hematopoiesis for about three to four or three to six months following transplantation into a recipient. In particular embodiments, hematopoietic progenitor cells have the ability to differentiate into at least two different hematopoietic cells.

The cells of the second population may be transduced or not transduced. In certain embodiments, the cells of the second population are transduced with a transfer vector that expresses a therapeutic polypeptide, since it may be advantageous for at least a portion of these cells to express the therapeutic polypeptide during the time period when cells of the second population repopulate the transplant recipient. It particular embodiments, the transduced cells of the second population are not subjected to puromycin. Therefore, while in certain embodiments, the same therapeutic polypeptide is expressed by transduced cells in the first and second cell populations, the same or different transfer vectors may be used to transduce each of the first and second cell populations. In specific embodiments, the transfer vector used to transduce the first population of cells comprises a polynucleotide encoding a puromycin resistance gene, while the transfer vector used to transduce the second population of cells may either include such a polynucleotide or not.

Accordingly, in certain embodiments, the present invention provides a method of reducing or inhibiting myelosuppression that comprises providing to a subject in need thereof both: (1) a cell population comprising transduced stem cells or multipotent cells that was produced and puromycin-selected as described above, and (2) another population of cells having a reduced percentage of stem cells as compared to the third population of cells, which, in particular embodiments, may have been prepared as described herein. For example, this other population of cells may have been exposed to conditions that induce at least partial differentiation of stem cells. This other population of cells may be either transduced or not transduced, and it be may be contacted with puromycin or not. This other population of cells may comprise hematopoietic progenitor cells. Either or both population of cell may be produced from cells obtained from bone marrow, peripheral mobilized blood, cord blood, or embryonic stem cells.

Similarly, in certain embodiments, the present invention provides a highly related method of reducing or inhibiting myelosuppression following transplantation of transduced stem cells into a transplant recipient, comprising transplanting a first population of cells comprising transduced stem cells into a transplant recipient in combination with a second population of cells having a reduced percentage of stem cells as compared to the first population of cells, wherein said first population of cells was produced by a procedure comprising selecting for transduced cells, wherein said selection comprises contacting the first population of cells with 1-25 μg/ml puromycin for 4 days or less, wherein said first population of cells was previously contacted with a transfer vector comprising a polynucleotide sequence encoding a puromycin resistance polypeptide operably linked to a promoter sequence, under conditions and for a time sufficient to permit integration of said polynucleotide into the genome of a plurality of cells within said first population of cells.

In particular embodiments, the first population of cells comprises stem cells transduced with a transfer vector comprising a polynucleotide encoding a therapeutic polypeptide. Accordingly, these methods may be used in the treatment of a variety of diseases and disorders, including any of those described infra, and in particular embodiments, diseases of the hematopoietic system, wherein said treatment includes myeloblation of a transplant recipient's endogenous bone marrow or hematopoietic cells. In particular embodiments, the first and second population of cells comprise cells capable of differentiating into one or more hematopoietic cells, such as, e.g., red blood cells. In particular embodiments, the first population of cells has the ability to engraft in the transplant recipient and provide long-term repopulation of a cellular compartment, e.g., hematopoietic cells, whereas the second population of cells has the ability to engraft in the transplant recipient and provide short-term or transient repopulation of a cellular compartment, e.g., hematopoietic cells.

In one embodiment, the present invention includes a method of inhibiting myelosuppression following transplantation of transduced multipotent cells into a transplant recipient, comprising transplanting a first population of cells comprising transduced multipotent cells into a transplant recipient in combination with a second population of cells having a reduced percentage of multipotent cells as compared to the first population of cells, wherein said second population of cells is capable of transiently repopulating the transplant recipient. In particular embodiments, said first population of cells comprises multipotent cells transduced with a transfer vector comprising a polynucleotide encoding a therapeutic polypeptide. In various embodiments, said second population cells is transduced or is not transduced. In certain embodiments, said first population of cells is capable of producing at least two distinct cell lineages for a duration of at least four months in vivo after transplantation of said first population of cells into the transplant recipient. In certain embodiments, said second population of cells is capable of transiently repopulating the hematopoietic system of the transplant recipient. In particular embodiments, said transplant recipient has undergone myeloablative therapy prior to said transplanting. In certain embodiments, said first and second populations of cells comprise hematopoietic cells. In particular embodiments, said second population of cells has a reduced percentage of multipotent cells as compared to the first population of cells. In particular embodiments, said second population of cells was exposed to conditions that induce expansion and/or at least partial differentiation of multipotent cells prior to said transplanting. In certain embodiments, said second population of cells was expanded in culture prior to said transplanting. In various embodiments, said first and second populations of cells were obtained from the transplant recipient. In certain embodiments, said first and/or second population of cells were obtained from bone marrow, peripheral mobilized blood, cord blood, and/or embryonic stem cells. In certain embodiments, said first population of cells was produced by a procedure comprising selecting for transduced cells, wherein said procedure comprises contacting the first population of cells with 1-25 μg/ml puromycin for 4 days or less, wherein said first population of cells was previously contacted with a transfer vector comprising a polynucleotide sequence encoding a puromycin resistance polypeptide operably linked to a promoter sequence, under conditions and for a time sufficient to permit integration of said polynucleotide into the genome of a plurality of cells within said first population of cells.

In particular embodiments of the present invention, any of the methods described above under the heading “Methods of Producing and Selecting Transduced Cells, and Related Methods of Enhancing the Reconstitution of Cell Populations in a Transplant Recipient by Transduced Cells and Delivering a Therapeutic Polypeptide to a Subject in Need Thereof” may be combined with any of the methods described herein under the heading “Methods of Reducing Myelosuppression in Transplant Recipients,” thus providing particular embodiments for enhancing the reconstitution of transplanted, transduced stem cells while reducing myelosuppression.

Accordingly, in one embodiment, the present invention provides a method of transplanting transduced stem cells into a transplant recipient, said method comprising: (i) contacting in vitro a first population of cells comprising multipotent cells, including stem cells, with a transfer vector comprising a polynucleotide sequence encoding a puromycin resistance polypeptide operably linked to a promoter sequence, thereby generating a second population of cells comprising transduced multipotent cells, including stem cells; (ii) contacting in vitro said second population of cells with puromycin at a concentration of 1-25 μg/ml for 4 days or less, thereby generating a third population of cells comprising transduced multipotent cells, including stem cells, wherein said third population of cells comprises a higher percentage of transduced multipotent cells than said second population of cells, and wherein said third population of cells is capable of sustaining the production of at least two distinct cell lineages containing said transfer vector for a duration of at least four months in vivo after transplantation of said third population of cells into a transplant recipient; (iii) transplanting into said transplant recipient a plurality of said third population of cells in combination with a fourth population of cells, said fourth population of cells comprising progenitor cells, wherein said fourth population of cells is capable of providing short term hematopoietic support after transplantation of said fourth population of cells into the transplant recipient. It is understood that when said third and fourth population of cells are transplanted in combination, this does not necessarily mean that they are transplanted simultaneously; instead, one of the two populations may be transplanted prior to, at the same time as, or after transplantation by the other population. However, both populations will be present in the transplant recipient during a time period, and, in certain embodiments, they may be transplanted at the same time, within one hour, within two hours, or within twenty-four hours of each other.

It is understood that the fourth population of cells, which is transplanted to provide transient or short term repopulation, may have any of the characteristics described above for cell populations intended to provide transient or short term repopulation. Thus, in particular embodiments, said fourth population of cells was previously exposed to conditions that induce expansion and/or at least partial differentiation of multipotent cells. In various embodiments, said fourth population of cells is not transduced or it is transduced. Thus, the fourth population of cells may or may not comprise transduced cells. In one embodiment, the fourth population of cells comprises cells transduced and selected by a method comprising: (i) contacting the fourth population of cells with a transfer vector comprising a polynucleotide sequence encoding a puromycin resistance polypeptide operably linked to a promoter sequence; and (ii) contacting the fourth population of cells with puromycin at a concentration of 1-25 μg/ml for 4 days or less, thereby selecting for transduced cells comprising the puromycin resistance polypeptide.

In particular embodiments, said fourth population of cells comprises hematopoietic cells. In certain embodiments, said first and fourth population of cells were obtained from the same subject. In particular embodiment, said first and fourth population of cells were obtained from bone marrow, peripheral mobilized blood, cord blood, and/or embryonic stem cells.

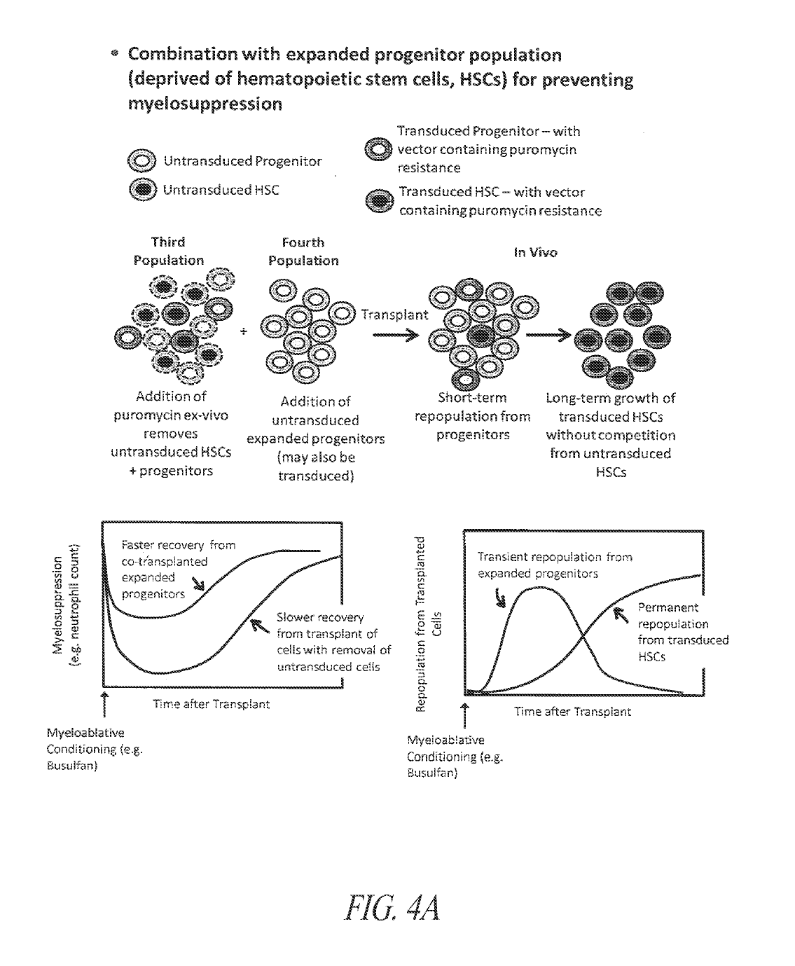

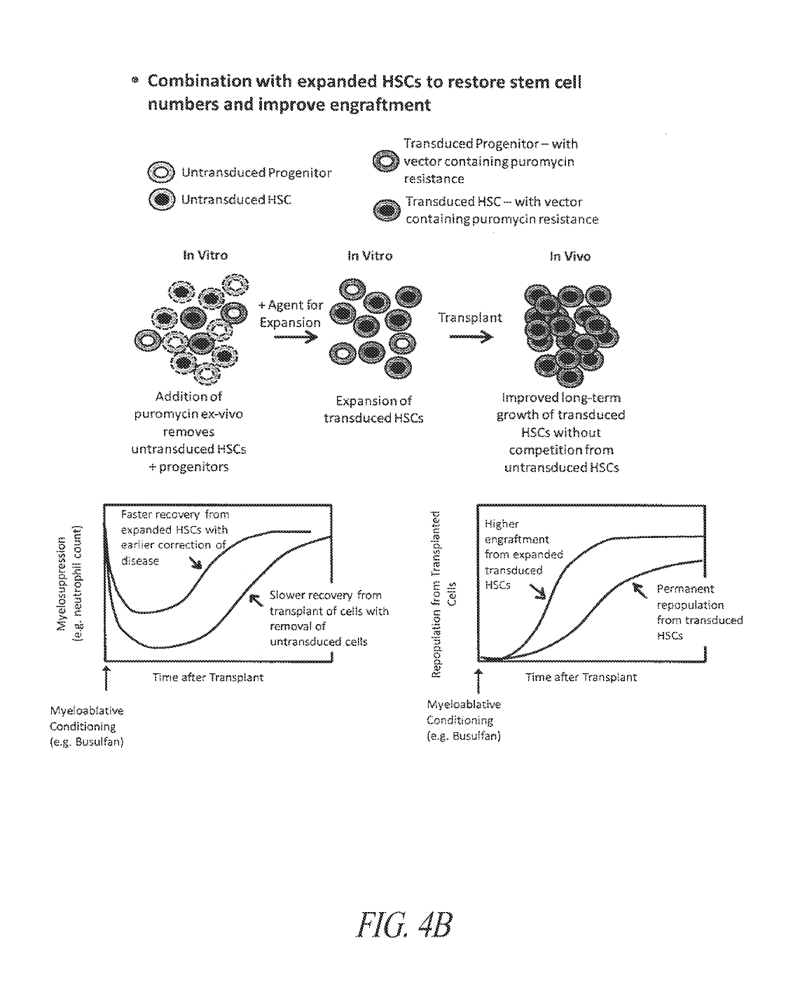

FIG. 4A provides a schematic diagram showing a method of the present invention for improving engraftment that includes both: (1) the selection of cells, including multipotent cells, such as hematopoietic stem cells (HSCs), transduced with a transfer vector that confers puromycin resistance; and (2) the transplant of both the selected transduced cells, which include multipotent cells such as HSCs, in combination with untransduced expanded progenitor cells. Following transplant, the untransduced expanded progenitor cells provide short-term repopulation, while the selected transduced HSCs grow without competition from untransduced HSCs and eventually reconstitute a cell population within the recipient. The graphs at the bottom of the figure show the level of myelosuppression over time following transplant into a recipient after myeloablation (left graph), and the repopulation from transplanted cells over time following transplant into the recipient after myeloablation (right graph). As shown, it is expected that the transplant recipient will exhibit a faster recovery when transplanted with transduced HSCs in combination with expanded progenitor cells, as compared to a slower recovery following transplant of transduced cells alone, following the removal of untransduced cells. In addition, it is expected that the transplant recipient will transient repopulation from the expanded progenitor cells, and permanent repopulation from the transduced HSCs.

FIG. 4B provides a schematic diagram showing a method of the present invention for improving engraftment that includes both: (1) the selection of cells, including multipotent cells, such as hematopoietic stem cells (HSCs), transduced with a transfer vector that confers puromycin resistance; and (2) the expansion of the selected transduced cells by culturing them in the presence of an agent that promotes the expansion of HSCs, such as the aryl hydrogen receptor antagonist, SR1. These expanded and transduced cells are transplanted into a recipient, where the transduced multipotent cells grow without competition from untransduced cells and eventually reconstitute a cell population within the recipient. The graphs at the bottom of the figure show the level of myelosuppression over time following transplant into a recipient after myeloablation (left graph), and the repopulation from transplanted cells over time following transplant into the recipient after myeloablation (right graph). As shown, it is expected that the transplant recipient will exhibit a faster recovery when transplanted with expanded HSCs, with accompanying earlier correction of disease, as compared to a slower recovery following transplant of non-expanded cells, following the removal of untransduced cells. In addition, it is expected that the transplant recipient will display higher levels of engraftment and permanent repopulation when transplanted with expanded transduced HSCs, as compared to a lower repopulation following transplant with non-expanded cells, following selection of transduced cells.

Transfer Vectors

The present invention further provides transfer vectors, which may be used to practice methods of the present invention. These vectors are designed to express a puromycin resistance polypeptide, thereby facilitating the selection of transduced cells. In preferred embodiments, the transfer vector is further designed to express a therapeutic polypeptide

While the skilled artisan will appreciate that such transfer vectors may be produced using a variety of different viral vectors, in particular embodiments, the transfer vector is a retroviral vector or a lentiviral vector, in part since lentiviral vectors are capable of providing efficient delivery, integration and long term expression of transgenes into non-dividing cells both in vitro and in vivo. A variety of lentiviral vectors are known in the art, see Naldini et al., (1996a, 1996b, and 1998); Zufferey et al., (1997); Dull et al., 1998, U.S. Pat. Nos. 6,013,516; and 5,994,136, any of which may be adapted to produce a transfer vector of the present invention. In general, these vectors are plasmid-based or virus-based, and are configured to carry the essential sequences for transfer of a nucleic acid encoding a therapeutic polypeptide into a host cell.

The lentiviral genome and the proviral DNA include three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3′ LTR's serve to promote transcription and polyadenylation of the virion RNAs, respectively. Lentiviruses have additional genes including vif, vpr, tat, rev, vpu, nef and vpx. Adjacent to the 5′ LTR are sequences necessary for reverse transcription of the genome (the tRNA primer binding site) and for efficient encapsidation of viral. RNA into particles (the Psi site).

In further embodiments, the lentiviral vector is an HIV vector. Thus, the vectors may be derived from human immunodeficiency-1 (HIV-1), human immunodeficiency-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), caprine arthritis encephalitis virus (CAEV) and the like. HIV based vector backbones (i.e., HIV cis-acting sequence elements and HIV gag, pol and rev genes) are generally be preferred in connection with most aspects of the present invention in that HIV-based constructs are the most efficient at transduction of human cells.

In a particular embodiment, the transfer vector of the invention comprises a left (5′) retroviral LTR; a retroviral export element, optionally a lentiviral Rev response element (RRE); a promoter, or active portion thereof, (and optionally a locus control region (LCR), or active portion thereof), operably linked to a gene of interest (e.g., encoding a therapeutic polypeptide); a polynucleotide sequence encoding a puromycin resistance polypeptide operably linked to a promoter; and a right (3′) retroviral LTR. The transfer vector of the invention can further comprise other elements, such as one or more of a central polypurine tract/DNA flap (cPPT/FLAP), including, for example, a psi packaging signal and/or a cPPT/FLAP from HIV-1.

In certain embodiment, the promoter of the 5′ LTR is replaced with a heterologous promoter, including, for example, cytomegalovirus (CMV) promoter.

In one embodiment of the invention, an LTR region, such as the 3′ LTR, of the vector is modified in the U3 and/or U5 regions, wherein a self-inactivating (SIN) vector is created. Such modifications contribute to an increase in the safety of the vector for gene delivery purposes. In one embodiment, the SIN vector of the invention comprises a deletion in the 3′ LTR wherein a portion of the U3 region is replaced with an insulator element. Exemplary SIN vectors are described, e.g., in U.S. Patent Application Publication No. 2006/0057725, and U.S. Pat. Nos. 7,250 and 6,165,782. The insulator prevents the enhancer/promoter sequences within the vector from influencing the expression of genes in the nearby genome, and vice/versa, to prevent the nearby genomic sequences from influencing the expression of the genes within the vector. Exemplary insulator sequences are described, e.g., in U.S. Patent Application No. 2006/0057725 and U.S. Pat. No. 5,610,053. In a further embodiment of the invention, the 3′ LTR is modified such that the U5 region is replaced, for example, with an ideal poly(A) sequence. It should be noted that modifications to the LTRs such as modifications to the 3′ LTR, the 5′ LTR, or both 3′ and 5′ LTRs, are also included in the invention.

In a particular embodiment, the transfer vector of the invention comprises a left (5′) retroviral LTR; a retroviral export element, optionally a lentiviral Rev response element (RRE); a promoter, or active portion thereof, and a locus control region (LCR), or active portion thereof, operably linked to a gene of interest; a polynucleotide sequence encoding a puromycin resistance polypeptide operably linked to a promoter; and a right (3′) retroviral LTR. The retroviral vector of the invention can further comprise a central polypurine tract/DNA flap (cPPT/FLAP), including, for example, a cPPT/FLAP from HIV-1. In another embodiment, the promoter of the 5′ LTR is replaced with a heterologous promoter, including, for example, cytomegalovirus (CMV) promoter. In particular embodiments, the U5 region of the left (5′) LTR, the right (3′) LTR, or both the left and right LTRs are modified to replace all or a portion of the region with an ideal poly(A) sequence and the U3 region of the left (5′) long terminal repeat (LTR), the right (3′) LTR, or both the left and right LTRs are modified to include one or more insulator elements. In one embodiment the U3 region is modified by deleting a fragment of the U3 region and replacing it with an insulator element. In specific embodiments, the U5 region of the right (3′) LTR is modified by deleting the U5 region and replacing it with a DNA sequence, for example an ideal poly(A) sequence. In still another embodiment, the vector comprises an insulator element comprising an insulator from an β-globin locus, including, for example, chicken HS4.

Transfer vectors of the present invention comprise a polynucleotide sequence that encodes a puromycin resistance polypeptide, or a functional variant or fragment thereof. Typically, a functional variant comprises at least 90%, at least 95%, or at least 99% amino acid identity to a puromycin resistance polypeptide, and a functional fragment comprises a portion of the puromycin resistance polypeptide sufficient to confer resistance to puromycin. Such functional variants and fragments may confer at least 50%, at least 60%, at least 70%, at least 80%, or at least 90% puromycin resistance activity as the wild-type puromycin resistance polypeptide from which they were derived, in cells where they are expressed. Puromycin resistance activity may be readily determined by one of skill in the art. In certain embodiments, the puromycin resistance gene is the pac gene, or a codon-optimized variant thereof, which encodes a Puromycin N-acetyl-transferase (PAC), or a functional variant or fragment thereof.

Transfer vectors, including lentiviral vectors, of the invention may comprise a gene of interest, including, for example, polynucleotide sequences that express a polypeptide of interest or a therapeutic polypeptide. In particular embodiments, a therapeutic polypeptide is provided to a patient in whom said polypeptide is expressed at a reduced level or in a mutated, less functional form. Examples of genes of interest and their expressed polypeptides include, but are not limited to: the adrenoleukodystrophy (ALD) gene or coding regions thereof, and the ALD protein, as described in U.S. Pat. No. 5,644,045; a globin gene or a gene which encodes an antisickling protein. In one embodiment, the globin gene expressed in the retroviral vector of the invention is β-globin, δ-globin, or γ-globin. In another embodiment, the human β-globin gene is the wild type human β-globin gene or human β^{A}-globin gene. In another embodiment, the human β-globin gene comprises one or more deletions of intron sequences or is a mutated human β-globin gene encoding at least one antisickling amino acid residue. Antisickling amino acids can be derived from human δ-globin or human γ-globin. In another embodiment, the mutated human β-globin gene encodes a threonine to glutamine mutation at codon 87 (β^{A}-T87Q). Examples of globin sequences that may be used according to the invention are also provided in U.S. Pat. No. 5,861,488, and exemplary transfer vectors comprising globin sequences are also described in U.S. Pat. No. 5,631,162.

The promoter(s) of the transfer vector can be one which is naturally (i.e., as it occurs with a cell in vivo) or non-naturally associated with the 5′ flanking region of a particular gene. Promoters can be derived from eukaryotic genomes, viral genomes, or synthetic sequences. Promoters can be selected to be non-specific (active in all tissues), tissue specific, regulated by natural regulatory processes, regulated by exogenously applied drugs, or regulated by specific physiological states such as those promoters which are activated during an acute phase response or those which are activated only in replicating cells. Non-limiting examples of promoters in the present invention include the retroviral LTR promoter, cytomegalovirus immediate early promoter, SV40 promoter, dihydrofolate reductase promoter, and cytomegalovirus (CMV). The promoter can also be selected from those shown to specifically express in the select cell types which may be found associated with conditions including, but not limited to, hemoglobinopathies. In one embodiment of the invention, the promoter is cell specific such that gene expression is restricted to red blood cells. Erythrocyte-specific expression can be achieved by using the human β-globin promoter region and locus control region (LCR).

The polynucleotide encoding the puromycin resistance polypeptide and the polynucleotide encoding the therapeutic polypeptide may be operably linked the same or different promoter sequences. The puromycin resistance polypeptide and/or the therapeutic polypeptide may be expressed from one or more constitutive promoters. However, transfer vectors of the invention may contain one or more promoter or enhancer elements that allow for temporal or tissue-specific expression of one or both the therapeutic polypeptide and/or the puromycin resistance gene. In particular embodiments, it may be desired to express a therapeutic polypeptide only in those cells where it is naturally expressed in a mammal. Therefore, a polynucleotide encoding a therapeutic polypeptide may be operably linked to a tissue-specific promoter and/or enhancer that selectively drives expression of the therapeutic polypeptide in those cells. One example of a tissue-specific promoter that may be used to drive expression in red blood cells is the β-globin promoter and LCR.

In certain embodiments, it may be desirable to express the puromycin resistance polypeptide using an inducible promoter, so that this polypeptide will not be expressed, or only expressed negligibly following introduction of cells transduced with the transfer vector into a subject. For example, it may be desirable to induce expression of the puromycin resistance polypeptide following transduction and prior to or during the puromycin selection process. In other embodiments, it may be desirable to preferentially express the puromycin resistance gene in stem cells as compared to progenitor or differentiated cells, in order to enhance selection of transduced stem cells, which may be advantageous for implantation and reconstitution. Thus, in the transfer vector, the polynucleotide encoding the puromycin resistance polypeptide may be operably linked to an inducible promoter and/or enhancer or a promoter and/or enhancer more active in stem cells than progenitor or differentiated cells.

A variety of inducible promoters and/or enhancer that may be used are known in the art, including but not limited to, e.g., the Cre/loxP system, two tetracycline-responsive Tet systems (Tet-On, Tet-Off), the glucocorticoid-responsive mouse mammary tumor virus promoter (MMTVprom), the ecdysone-inducible promoter (EcP), and the T7 promoter/T7 RNA polymerase system (T7P).

In one specific embodiment, the HIV-based recombinant transfer vector contains, in a 5′ to 3′ direction, the 5′ flanking HIV LTR, a packaging signal or psi+, a central polypurine tract or DNA flap of HIV-1 (cPPT/FLAP), a Rev-response element (RRE), the human β-globin gene 3′ enhancer, a gene of interest, such as the human-globin gene variant containing the β^{A87 Thr:Gln} mutation, 266 bp of the human β-globin promoter, 2.7 kb of the human β-globin LCR, the PGK promoter or an inducible promoter, a polynucleotide that encodes a puromycin resistance polypeptide, a polypurine tract (PPT), and the 3′ flanking HIV LTR. The LTR regions further comprise a U3 and U5 region, as well as an R region. The U3 and U5 regions can be modified together or independently to create a transfer vector which is self-inactivating, thus increasing the safety of the vector for use in gene delivery. The U3 and U5 regions can further be modified to comprise an insulator element. In one embodiment of the invention, the insulator element is chicken HS4.

In other embodiments, transfer vectors of the present invention express both puromycin resistance polypeptide and a suicide gene product, e.g., using the conditional suicide gene herpes simplex virus-thymidine kinase. In addition, they can express a therapeutic polypeptide or gene of interest. Thus, neoplastic cells that may be generated upon vector-mediated insertional mutagenesis in the transplant recipient would be rendered treatable by chemically-induced cell suicide (e.g., ganciclovir; Naujok O. et al. Stem Cell Rev. 2010 September; 6(3):450-61). Other conditional suicide gene may be used in lieu of herpes simplex virus-thymidine kinase encoding gene. In particular embodiments, the puromycin resistance and the conditional cell suicide proteins are co-expressed by means of (i) an “internal ribosomal entry site (IRES)” within a polycistronic vector, (ii) by cleavage of a precursor protein or (iii) as a fusion protein.

Thus, in another embodiment of the invention, the transfer vector includes a polynucleotide sequence comprising a promoter operably linked to a suicide gene. In a particular embodiment, the suicide gene is HSV thymidine kinase (HSV-Tk). The transfer vector can also include a polynucleotide comprising a gene for in vivo selection of the cell, such as a gene for in vivo selection, e.g., a methylguanine methyltransferase (MGMT) gene. In particular embodiments, the suicide gene is operably linked to a constitutive or an inducible promoter, including any of those described herein. In one embodiment, the polynucleotide sequence comprising a promoter operably linked to a suicide gene is present in the vector downstream of the 5′ LTR and downstream of the polynucleotide encoding the therapeutic protein. In certain embodiments, the polynucleotide sequence comprising a promoter operably linked to a suicide gene is orientated so that the 5′ end of the promoter operably linked to the suicide gene is located towards the 5′ end of the polynucleotide encoding the therapeutic protein (the polynucleotide encoding the therapeutic protein is in the reverse orientation compared the polynucleotide sequence comprising a promoter operably linked to a suicide gene; thus the 5′ end of the polynucleotide encoding the therapeutic protein is closer to the 3′ LTR than the 5′ LTR) and the 3′ end of the polynucleotide sequence comprising a promoter operably linked to a suicide gene is located towards the 5′ end of the ppt and/or 3′ LTR.

In certain embodiments, the polynucleotide encoding the suicide gene is orientated in the vector such that its expression is not driven by a promoter in the vector. Thus, the polynucleotide encoding the suicide protein is not operably linked to a promoter within the vector. Rather, expression of the suicide gene occurs if the vector inserts into a region of chromosomal DNA of a cell under the influence of a cellular promoter. In particular embodiments, the suicide protein may be either conditional or constitutive. In one embodiment, the polynucleotide encoding the suicide protein is present in the vector downstream of the 5′ LTR and upstream of the polynucleotide encoding the therapeutic protein. In certain embodiments, the polynucleotide encoding the suicide protein is orientated so that the 5′ end of the polynucleotide encoding the suicide protein is located towards the 5′ LTR, and the 3′ end of the polynucleotide encoding the suicide protein is located towards the 3′ end of the polynucleotide encoding the therapeutic protein. Thus, the polynucleotide encoding the suicide protein and the polynucleotide encoding the therapeutic protein may be in the opposite orientation. In particular embodiments of the vector where these elements are present, the polynucleotide encoding the suicide protein may be located in the vector upstream of the cPPT/FLAP and/or RRE elements. In particular embodiments, wherein the polynucleotide encoding the suicide protein is downstream of the 5′ LTR and upstream of the cPPT/FLAP and/or RRE elements, a splice acceptor sequence may be included 5′ to the suicide protein, e.g., directly adjacent to the polynucleotide encoding the suicide protein. In certain embodiments, the splice acceptor sequence is 20 bases, 10 bases, 5 bases or fewer bases upstream of the polynucleotide encoding the suicide protein.

In one embodiment, the transfer vector comprises a polynucleotide encoding the suicide protein that is not operably linked to a promoter within the vector, wherein the polynucleotide encoding the suicide protein is downstream of the 5′ LTR and upstream of the cPPT/FLAP and/or RRE elements, and wherein a splice acceptor site is included 5′ to the suicide protein, e.g., directly adjacent to the polynucleotide encoding the suicide protein or within 20 bases, 10 bases, 5 bases or fewer bases upstream of the polynucleotide encoding the suicide protein.

In certain embodiments, the puromycin resistance protein and the suicide protein are expressed as an in-frame fusion protein or polypeptide. In particular embodiments, they are expressed as a direct fusion of the puromycin resistance polypeptide and the suicide polypeptide. In certain embodiments, the fusion polypeptide comprises a linker sequence between the puromycin resistance polypeptide and the suicide polypeptide.

A peptide linker sequence may be employed to separate any two or more polypeptide components by a distance sufficient to ensure that each polypeptide folds into its appropriate secondary and tertiary structures so as to allow the polypeptide domains to exert their desired functions. Such a peptide linker sequence is incorporated into the fusion polypeptide using standard techniques in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. Linker sequences are not required when a particular fusion polypeptide segment contains non-essential N-terminal amino acid regions that can be used to separate the functional domains and prevent steric interference. Preferred linkers are typically flexible amino acid subsequences which are synthesized as part of a recombinant fusion protein. Linker polypeptides can be between 1 and 200 amino acids in length, between 1 and 100 amino acids in length, or between 1 and 50 amino acids in length, including all integer values in between.

Exemplary linkers include, but are not limited to the following amino acid sequences: GGG; DGGGS (SEQ ID NO:16); TGEKP (SEQ ID NO:17) (see, e.g., Liu et al., PNAS 5525-5530 (1997)); GGRR (SEQ ID NO:18) (Pomerantz et al. 1995, supra); (GGGGS)ₙ (SEQ ID NO:19) (Kim et al., PNAS 93, 1156-1160 (1996.); EGKSSGSGSESKVD (SEQ ID NO:20) (Chaudhary et al., 1990, Proc. Natl. Acad. Sci. U.S.A. 87:1066-1070); KESGSVSSEQLAQFRSLD (SEQ ID NO:21) (Bird et al., 1988, Science 242:423-426), GGRRGGGS (SEQ ID NO:22); LRQRDGERP (SEQ ID NO:23); LRQKDGGGSERP (SEQ ID NO:24); LRQKd(GGGS)₂ ERP (SEQ ID NO:25). Alternatively, flexible linkers can be rationally designed using a computer program capable of modeling both DNA-binding sites and the peptides themselves (Desjarlais & Berg, PNAS 90:2256-2260 (1993), PNAS 91:11099-11103 (1994) or by phage display methods.

In certain embodiments, the linker sequence comprises a Gly3 linker sequence, which includes three glycine residues.

In particular embodiments, the linker sequence is cleavable. In particular embodiments, the linker sequence comprises an autocatalytic peptide cleavage site. Exemplary polypeptide cleavage signals include polypeptide cleavage recognition sites such as protease cleavage sites, nuclease cleavage sites (e.g., rare restriction enzyme recognition sites, self-cleaving ribozyme recognition sites), and self-cleaving viral oligopeptides (see deFelipe and Ryan, 2004. Traffic, 5(8); 616-26).

Suitable protease cleavages sites and self-cleaving peptides are known to the skilled person (see, e.g., in Ryan et al., 1997. J. Gener. Virol. 78, 699-722; Scymczak et al. (2004) Nature Biotech. 5, 589-594). Exemplary protease cleavage sites include, but are not limited to the cleavage sites of potyvirus NIa proteases (e.g., tobacco etch virus protease), potyvirus HC proteases, potyvirus P1 (P35) proteases, byovirus NIa proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PYVF (parsnip yellow fleck virus) 3C-like protease, heparin, thrombin, factor Xa and enterokinase. Due to its high cleavage stringency, TEV (tobacco etch virus) protease cleavage sites are preferred in one embodiment, e.g., EXXYXQ(G/S) (SEQ ID NO:3), for example, ENLYFQG (SEQ ID NO:4) and ENLYFQS (SEQ DI NO:5), wherein X represents any amino acid (cleavage by TEV occurs between Q and G or Q and S).

In a particular embodiment, self-cleaving peptides include those polypeptide sequences obtained from potyvirus and cardiovirus 2A peptides, FMDV (foot-and-mouth disease virus), equine rhinitis A virus, Thosea asigna virus and porcine teschovirus.

In certain embodiments, the self-cleaving polypeptide site comprises a 2A or 2A-like site, sequence or domain (Donnelly et al., 2001. J. Gen. Virol. 82:1027-1041).

Exemplary 2A sites include the following sequences:

SEQ ID NO: 6
LLNFDLLKLAGDVESNPGP
SEQ ID NO: 7
TLNFDLLKLAGDVESNPGP
SEQ ID NO: 8
LLKLAGDVESNPGP
SEQ ID NO: 9
NFDLLKLAGDVESNPGP
SEQ ID NO: 10
QLLNFDLLKLAGDVESNPGP
SEQ ID NO: 11
APVKQTLNFDLLKLAGDVESNPGP
SEQ ID NO: 12
VTELLYRMKRAETYCPRPLLAIHPTEARHKQKIVAPVKQT
SEQ ID NO: 13
LNFDLLKLAGDVESNPGP
SEQ ID NO: 14
LLAIHPTEARHKQKIVAPVKQTLNFDLLKLAGDVESNPGP
SEQ ID NO: 15
EARHKQKIVAPVKQTLNFDLLKLAGDVESNPGP

In one embodiment, the autocatalytic peptide cleavage site comprises a translational 2A signal sequence, such as, e.g., the 2A region of the aphthovirus foot-and-mouth disease virus (FMDV) polyprotein, which is an 18 amino acid sequence. Additional examples of 2A-like sequences that may be used include insect virus polyproteins, the NS34 protein of type C rotaviruses, and repeated sequences in Trypanosoma spp., as described, e.g., in Donnelly et al., Journal of General Virology (2001), 82, 1027-1041

In further embodiments, the transfer vector comprises “junk” sequence located between the polynucleotide sequence encoding the puromycin resistance polypeptide and the polynucleotide sequence comprising the suicide gene or cDNA. As used herein, the term “junk sequence” refers to a DNA sequence having no known function. In certain embodiments, the junk sequence does not have significant or detectable promoter or enhancer activity in mammalian cells, and it does not encode either any polypeptide or any polypeptide having any or any known functional activity. In certain embodiments, the polynucleotide sequence encoding junk sequence is flanked by a stop codon at its 5′ end and/or a start codon at its 3′ end.

In various embodiments, the transfer vector may comprise polynucleotides encoding a suicide protein and a puromycin resistance protein, and either the polynucleotide sequence encoding the suicide protein is upstream of the polynucleotide encoding a puromycin resistance protein, or vice versa. In one embodiment, the transfer vector comprises a polynucleotide sequence comprising a promoter operably linked to polynucleotide sequences encoding a suicide protein and a puromycin resistance protein. In particular embodiments, the polynucleotide sequences encoding a suicide protein and a puromycin resistance protein are operably linked to a constitutive or an inducible promoter, including any of those described herein.

In one embodiment, a polynucleotide sequence comprising a promoter operably linked to polynucleotide sequences encoding a suicide protein and a puromycin resistance protein is present in the vector downstream of the 5′ LTR and downstream of the polynucleotide encoding the therapeutic protein. In certain embodiments, the polynucleotide sequence comprising a promoter operably linked to polynucleotide sequences encoding a suicide protein and a puromycin resistance protein is orientated so that the 5′ end of the promoter is located towards the 5′ end of the polynucleotide encoding the therapeutic protein (the polynucleotide encoding the therapeutic protein is in the reverse orientation compared the polynucleotide sequence comprising a promoter operably linked to polynucleotide sequences encoding a suicide protein and a puromycin resistance protein; thus the 5′ end of the polynucleotide encoding the therapeutic protein is closer to the 3′ LTR than the 5′LTR) and the 3′ end of the polynucleotide sequence comprising a promoter operably linked to polynucleotide sequences encoding a suicide protein and a puromycin resistance protein is located towards the 5′ end of the ppt and/or 3′ LTR.

In one embodiment, the transfer vector comprises a splice acceptor sequence 5′ to the promoter operably linked to polynucleotide sequences encoding a suicide protein and a puromycin resistance protein, e.g., directly adjacent to the promoter. In certain embodiments, the splice acceptor sequence is 20 bases, 10 bases, 5 bases or fewer bases upstream of the promoter operably linked to polynucleotide sequences encoding a suicide protein and a puromycin resistance protein.

In additional embodiments, a splice acceptor sequence is included 5′ to the promoter operably linked to polynucleotide sequences encoding a suicide protein and a puromycin resistance protein and/or 5′ to the polynucleotide sequence encoding the suicide protein, and/or 5′ to the polynucleotide sequence encoding the puromycin protein, in any suitable combination thereof. The splice acceptor sequences can be 20 bases, 10 bases, 5 bases or fewer bases upstream of each of, or all of these polynucleotide sequences.

In certain embodiments, the polynucleotides encoding the suicide protein and puromycin resistance protein are orientated in the vector such that their expression is not driven by a promoter in the vector. Thus, the polynucleotides encoding the suicide protein and puromycin resistance protein are not operably linked to a promoter within the vector. Rather, expression of the polynucleotides encoding the suicide protein and puromycin resistance protein occurs if the vector inserts into a region of chromosomal DNA of a cell under the influence of a cellular promoter. In one embodiment, the polynucleotides encoding the suicide protein and puromycin resistance protein are present in the vector downstream of the 5′ LTR and upstream of the polynucleotide encoding the therapeutic protein. In certain embodiments, the polynucleotides encoding the suicide protein and puromycin resistance protein are orientated so that the 5′ end of the polynucleotide encoding the suicide protein or the puromycin resistance protein is located towards the 5′ LTR, and the 3′ end of the polynucleotide encoding the suicide protein or the puromycin resistance protein is located towards the 3′ end of the polynucleotide encoding the therapeutic protein. Thus, the polynucleotides encoding the polynucleotide encoding the suicide protein and the puromycin resistance protein may be in the opposite orientation to the polynucleotide encoding the therapeutic protein. In particular embodiments of the vector where these elements are present, the polynucleotides encoding the suicide protein and the puromycin resistance protein may be located in the vector upstream of the cPPT/FLAP and/or RRE elements. In particular embodiments, wherein the polynucleotides encoding the suicide protein and the puromycin resistance protein are downstream of the 5′ LTR and upstream of the cPPT/FLAP and/or RRE elements, a splice acceptor sequence may be included 5′ to the suicide protein, and/or the puromycin resistance protein e.g., directly adjacent to, or within 20 bases, 10 bases, 5 bases or fewer bases upstream of the polynucleotides encoding the suicide protein and/or the puromycin resistance protein.

In one embodiment, the transfer vector comprises polynucleotides encoding a suicide protein and a puromycin resistance protein that are not operably linked to a promoter within the vector, wherein the polynucleotides encoding a suicide protein and a puromycin resistance protein are downstream of the 5′ LTR and upstream of the cPPT/FLAP and/or RRE elements, and wherein a splice acceptor site is included 5′ to the suicide protein and/or the puromycin resistance protein, e.g., directly adjacent to, or within 20 bases, 10 bases, 5 bases or fewer bases upstream of the polynucleotides encoding the suicide protein and the puromycin resistance protein.

The transfer vector may comprise a splice acceptor sequence upstream of the promoter driving expression of the puromycin resistance polypeptide and/or suicide protein. In certain embodiments, the vector comprises a splice acceptor sequence directly upstream of the promoter sequence driving expression of the polynucleotide sequences encoding the puromycin resistance polypeptide and the suicide protein. In various embodiments, the transfer vector comprises polynucleotides encoding a suicide protein and a puromycin resistance protein, and either the polynucleotide sequence encoding the suicide protein is upstream of the polynucleotide encoding a puromycin resistance gene, or vice versa, and further comprises a splice acceptor sequence upstream of the promoter driving their expression.

In certain embodiments, the vector comprises a polynucleotide sequence encoding a puromycin resistance polypeptide upstream of a polynucleotide encoding a suicide protein, and further comprises a splice acceptor sequence upstream of the start codon of the polynucleotide that encodes the suicide protein or upstream of the start codon of the polynucleotide sequence that encodes the puromycin resistance polypeptide. In certain embodiments, the vector comprises a polynucleotide sequence encoding a suicide protein upstream of a polynucleotide encoding a puromycin resistance gene, and further comprises a splice acceptor sequence upstream of the start codon of the polynucleotide that encodes the suicide protein or upstream of the start codon of the polynucleotide sequence that encodes the puromycin resistance polypeptide.

In certain embodiments, the polynucleotide comprising the suicide gene or cDNA comprises a Kozak consensus sequence at the 5′ end of the suicide gene and a transcription terminator sequence 3′ of the suicide gene or cDNA. An exemplary strong Kozak sequence that may be used is the consensus sequence, (GCC)RCCATGG (SEQ ID NO:26), where R is a purine (A or G) (Kozak, 1986. Cell. 44(2):283-92, and Kozak, 1987. Nucleic Acids Res. 15(20):8125-48).

In particular embodiments, the transfer vector comprises an internal ribosome entry site (IRES) between the polynucleotide encoding the puromycin resistance polypeptide and the polynucleotide encoding the suicide protein. An IRES is a nucleotide sequence that allows for translation initiation in the middle of an mRNA. Accordingly, the presence of the IRES between the polynucleotide encoding the puromycin resistance polypeptide and the polynucleotide encoding the suicide protein allows the translation of separate puromycin resistance protein and suicide protein. A variety of IRES from viral genomes and mammalian RNAs are known and may be employed according to the present invention, including, e.g., the IRES from encephalomyocarditis virus (EMCV).

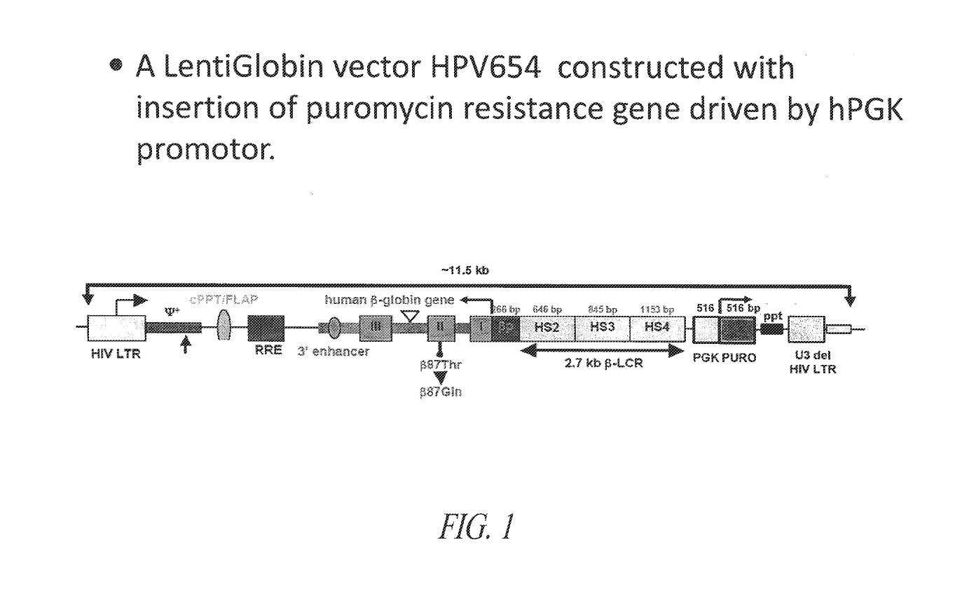

Transfer vectors may be made using routine molecular biology techniques known in the art. For example, the cDNA of the therapeutic gene of interest, such as, for example, human β-globin, is amplified by PCR from an appropriate library. The gene is cloned into a plasmid, such as pBluescript II KS (+) (Stratagene), containing a desired promoter or gene-expression controlling elements, such as the human β-globin promoter and LCR elements. Following restriction enzyme digestion, or other method known by one skilled in the art to obtain a desired DNA sequence, the nucleic acid cassette containing the promoter and LCR elements and therapeutic gene of interest is then inserted into an appropriate cloning site of the lentiviral vector, as shown in FIG. 1.

Transfer vectors, including lentiviral vectors, of the invention can be used in gene therapy, including for the treatment of hemoglobinopathies. The invention also includes host cells comprising, e.g., transfected with, the vectors of the invention. In one embodiment, the host cell is an embryonic stem cell, a somatic stem cell, or a progenitor cell.

In other embodiments, the invention provides methods for using the foregoing optimized vectors to achieve stable, high levels of gene expression in erythroid cells, e.g., in order to treat erythroid-specific diseases.

### EXAMPLES

### Example 1

### Enrichment of Transduced CD34 Cells by Puromycin Selection

This example demonstrates the successful transduction and selection of transduced CD34⁺ cells using a lentiviral vector that expresses a puromycin resistance gene, thereby producing a cell population enriched in transduced cells.

The lentiviral vector, HPV654, used in these experiments was constructed by inserting a polynucleotide sequence encoding a puromycin resistance polypeptide operably linked to the hPGK promoter, into a previously described vector that expresses a modified human β-globin polypeptide (β^{A}-T87Q-Globin Lentivirus, described in U.S. Patent Application Publication No. 2006/0057725. This modified human β^{A}-globin gene variant is mutated at codon 87 to encode a Glutamine [β^{A87} Thr:Gln (β^{A}-T87Q)], which is thought to be responsible for most of the antisickling activity of β-globin (Nagel et al. (1979) PNAS USA 767:670). A schematic diagram of the HPV654 vector is provided in FIG. 1. As shown in FIG. 1, the vector contains HIV LTR, HIV type-1 long terminal repeat ψ⁺; psi+ packaging signal; cPPT, central polypurine tract/DNA flap; RRE, Rev-responsive element; I, II, III, human β-globin gene exons; intervening sequence; globin promoter (from SnaBI to Cap site); the 3′ β globin enhancer (up to downstream AvrII site), and DNase I hypersensitive sites, HS2 (SmaI to XbaI), HS3 (SacI to PvuII) and HS4 (StuI to SpeI) of the LCR, PGK, human phosphoglycerate kinase promoter; puro, puromycin resistance gene ppt, polypurine tract; U3 del HIV LTR; and rabbit globin polyA.

Stocks of recombinant virus pseudotyped with vesicular stomatitis virus glycoprotein-G (VSV-G) were generated by transient transfection of 293T cells with the HPV654 vector together with separate plasmids expressing HIV-1 Gag-Pol, Rev, Tat and VSV-G. Virus was concentrated by ultracentrifugation at 4° C., and the viral pellet resuspended in StemPro-34 serum free medium (Life Technologies, Frederick, Md.). Viral titers were determined by qPCR analysis of transduced NIH3T3 cells with proviral copy number controls.

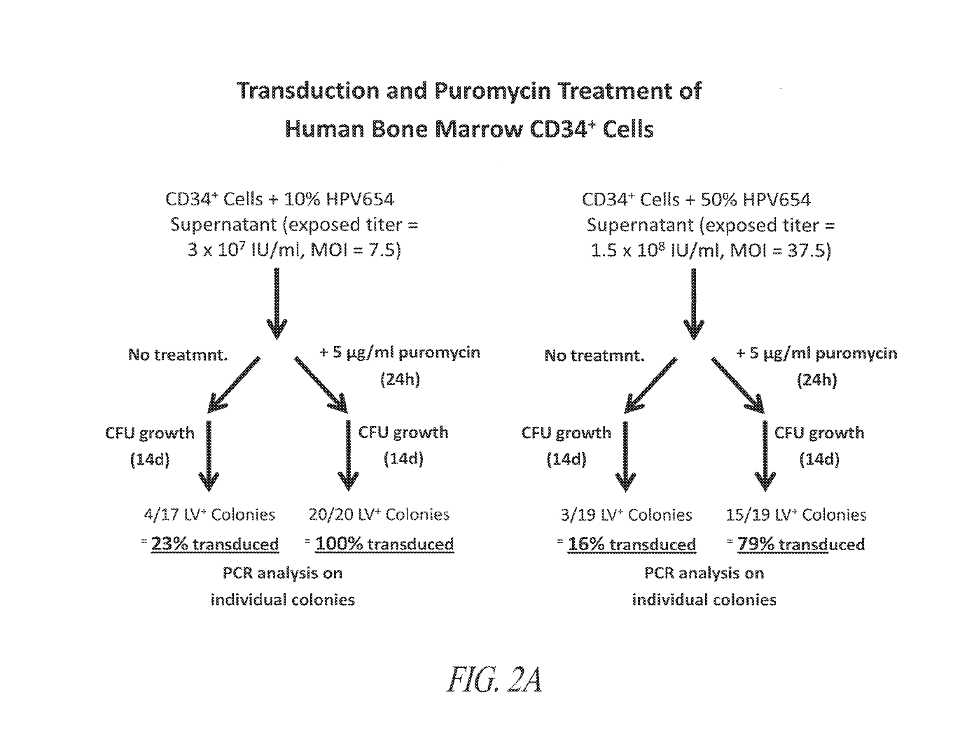

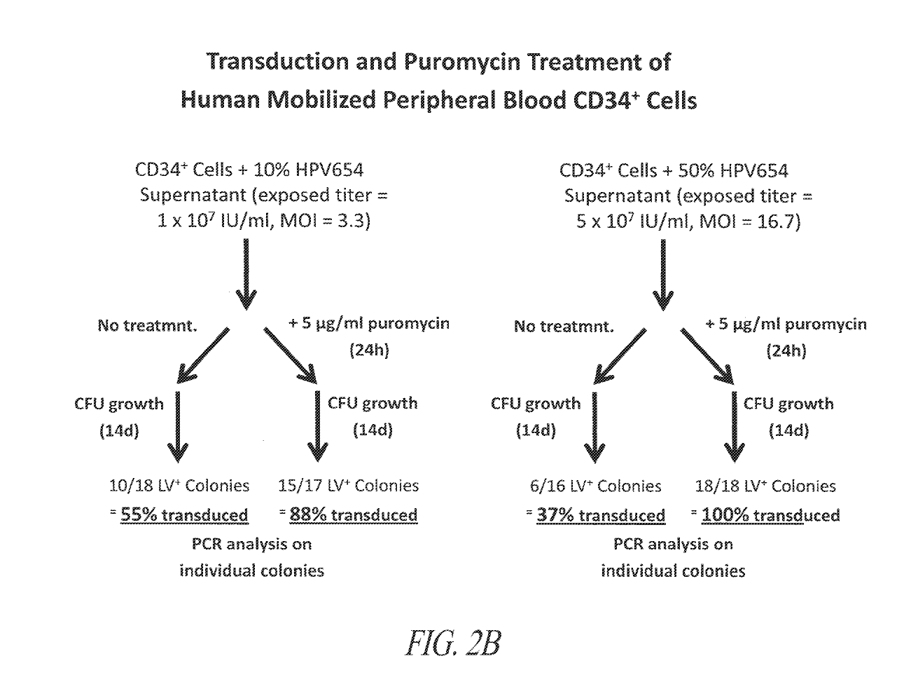

Either fresh bone marrow (BM, Lonza, Walkersville, Md.) or cryopreserved G-CSF-mobilized peripheral blood (mPB, AllCells, Emeryville, Calif.) CD34⁺ purified cells were used from normal human donors. At a cell concentration of 1×10⁶/ml, the CD34 cells were prestimulated for 24 hours (BM) or 18 hours (mPB) in StemPro-34 SFM supplemented with L-glutamine, 100 ng/ml hSCF, 100 ng/ml hTPO, 100 ng/ml hFLT3-L and 20 ng/ml hIL-3 for BM or 60 ng/ml hIL-3 for mPB. The cells were then resuspended at a concentration of 4×10⁶ (BM) or 3×10⁶ (mPB) cells/ml in the same medium containing cytokines with additional supplementation with 8 μg/ml protamine sulfate and the HPV654 supernatant at either 10% (final exposed titer of 3×10⁷ IU/ml with MOI of 7.5 for BM and 1×10⁷ IU/ml with MOI of 3.3 for mPB) or 50% (final exposed titer of 1.5×108 IU/ml with MOI of 37.5 for BM and 5×10⁷ IU/ml with MOI of 16.7 for mPB). At 24 hours after addition of HPV654 supernatant, fresh cytokine-containing medium was added to dilute cells to 2.0×10⁶/ml for BM or 1.5×10⁶/ml for mPB and cultured for further 24 hours, Each of the two infected cell populations was then divided into two samples; one sample from each cell population was treated with 5 μg/ml puromycin for 24 hours, and the other was not treated with puromycin, as depicted in FIG. 2. The cells were then washed for removal of puromycin and plated in MethoCult® H4434 (Stem Cell Technologies Inc., Vancouver, BC, Canada) at doses of 1×10³ and 4×10³ cells in 3 ml (puromycin untreated) and 4×10³ and 4×10⁴ (puromycin treated). Colony forming units-granulocyte/macrophage (CFU-GM) were then allowed to grow in culture for 14 days. Each colony was then analyzed for the presence of the lentiviral vector by PCR analysis of individual colonies using primers for erythropoietin gene for BM and human actin gene for mPB and primers specific for the lentiviral vector (GAG for BM and LTR for mPB). As shown in FIG. 2A, for the bone marrow CD34 cells transduced with 10% HPV654 supernatant, 4/17 (23%) colonies generated without puromycin selection were positive for the lentiviral vector, whereas 20/20 (100%) colonies generated with puromycin selection were positive for the lentiviral vector. For the cells transduced with 50% HPV654 supernatant, 3/19 (16%) colonies generated without puromycin selection were positive for the lentiviral vector, whereas 15/19 (79%) colonies generated with puromycin selection were positive for the lentiviral vector.

In the second experiment shown in FIG. 2B for G-CSF mobilized CD34 cells transduced with 10% HPV654 supernatant, 10/18 (55%) colonies generated without puromycin selection were positive for the lentiviral vector, whereas 15/17 (88%) colonies generated with puromycin selection were positive for the lentiviral vector. For the cells transduced with 50% HPV654 supernatant, 6/16 (37%) colonies generated without puromycin selection were positive for the lentiviral vector, whereas 18/18 (100%) colonies generated with puromycin selection were positive for the lentiviral vector.

These experiments demonstrate that enrichment of transduced CD34⁺ cell may be performed by puromycin selection, where the cells are contacted with puromycin for as little as 24 hours. Such selection may be used advantageously to produce cell populations comprising a high percentage of transduced cells, thereby enhancing subsequent engraftment and repopulation of transduced cells in a transplant recipient.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheetare incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of enhancing the reconstitution by transduced cells in a transplant recipient, said method comprising selecting transduced cells prior to transplantation into said transplant recipient, wherein said transduced cells are selected by a method comprising:
(i) contacting in vitro a first population of cells comprising multipotent cells, including stem cells, with a transfer vector comprising a polynucleotide sequence encoding a puromycin resistance polypeptide operably linked to a promoter sequence, thereby generating a second population of cells comprising transduced multipotent cells, including stem cells; and
(ii) contacting in vitro said second population of cells with puromycin at a concentration of 1-25 μg/ml for 4 days or less, thereby generating a third population of cells comprising transduced multipotent cells, including stem cells, wherein said third population of cells comprises a higher percentage of transduced multipotent cells than said second population of cells, and wherein said third population of cells is capable of sustaining the production of at least two distinct cell lineages containing said transfer vector for a duration of at least four months in vivo after transplantation of said third population of cells into a transplant recipient.

2. The method of claim 1, further comprising transplanting a plurality of said third population of cells into said transplant recipient.

3. The method of claim 1 or claim 2, wherein said first population of cells:
a) was obtained from said transplant recipient;
b) was obtained from bone marrow, peripheral mobilized blood, cord blood and/or embryonic stem cells; or
c) comprises hematopoietic stem cells.

4-7. (canceled)

8. The method of claim 1, wherein said transfer vector further comprises a polynucleotide sequence encoding a therapeutic polypeptide operably linked to a promoter sequence.

9. The method of claim 1, wherein said transfer vector is a retroviral vector, a lentiviral vector, a human immunodeficiency virus (HIV) vector, a simian immunodeficiency virus (SIV) vector, an equine infectious anaemia virus (EIAV) vector, or a transposon.

10-14. (canceled)

15. The method of claim 8, wherein the polynucleotide encoding the puromycin resistance polypeptide and the polynucleotide encoding the therapeutic polypeptide are operably linked to the same promoter sequence, or are operably linked to different promoter sequences.

16. (canceled)

17. The method of claim 15, wherein the promoter or promoters are selected form the group consisting of: a constitutive promoter, an inducible promoter, and a tissue specific promoter.

18-23. (canceled)

24. The method of claim 1 or claim 8, wherein said transfer vector further comprises a polynucleotide sequence comprising a suicide gene or cDNA, wherein said suicide gene or cDNA encodes a suicide polypeptide.

25. The method of claim 24, wherein said suicide gene or cDNA encodes a thymidine kinase derivative, a thymidylate kinase (TmpK) derivative, or a caspase derivative.

26-27. (canceled)

28. The method of claim 24, wherein said polynucleotide sequence comprising the suicide gene or cDNA is not operatively linked to a promoter sequence present in the transfer vector.

29-31. (canceled)

31. The method of claim 24, wherein the polynucleotide sequence comprising the suicide gene or cDNA and the polynucleotide sequence encoding the therapeutic polypeptide are present in the transfer vector in opposite orientations.

32. The method of claim 24, wherein said transfer vector comprises a splice acceptor sequence upstream of the suicide gene or cDNA.

33. (canceled)

34. The method of claim 24, wherein said transfer vector expresses said puromycin resistance polypeptide and said suicide polypeptide as an in-frame fusion polypeptide.

35-37. (canceled)

38. The method of claim 24, wherein said transfer vector comprises an internal ribosome entry site (IRES) between the polynucleotide sequence encoding the puromycin resistance polypeptide and the polynucleotide sequence comprising the suicide gene or cDNA.

39. The method of claim 34, wherein said fusion polypeptide comprises a linker sequence between the puromycin resistance polypeptide and the suicide polypeptide.

40. The method of claim 39, wherein said linker sequence comprises a Gly3 linker sequence, an autocatalytic peptide cleavage site, or a translational 2A signal sequence.

41-43. (canceled)

44. The method of claim 2, further comprising providing said third population of cells to a subject in combination with a fourth population of cells, said fourth population of cells comprising progenitor cells, wherein said fourth population of cells is capable of providing short term hematopoietic support after transplantation of said fourth population of cells into a transplant recipient.

45-46. (canceled)

47. The method of claim 44, wherein said fourth population of cells comprises cells transduced and selected by a method comprising:
(i) contacting the fourth population of cells with a transfer vector comprising a polynucleotide sequence encoding a puromycin resistance polypeptide operably linked to a promoter sequence; and
(ii) contacting the fourth population of cells with puromycin at a concentration of 1-25 μg/ml for 4 days or less, thereby selecting for transduced cells comprising the puromycin resistance polypeptide.

48-50. (canceled)

51. The method of claim 1, further comprising contacting at least one of said first, second or third population of cells with one or more agents capable of increasing the number of stem cells present in the contacted cell population.

52-56. (canceled)
